(19) United States
(12) Patent Application Publication
(10) Pub. No.: US 2020/0405650 A1
(43) Pub. Date: Dec. 31, 2020

(54) STARRY MESOPOROUS SILICA NANOPARTICLES AND SUPPORTED LIPID BI-LAYER NANOPARTICLES

(71) Applicants: Achraf Noureddine, Albuquerque, NM (US); Charles Jeffrey Brinker, Albuquerque, NM (US); Elizabeth Hjelvik, White Rock, NM (US)

(72) Inventors: Achraf Noureddine, Albuquerque, NM (US); Charles Jeffrey Brinker, Albuquerque, NM (US); Elizabeth Hjelvik, White Rock, NM (US)

(21) Appl. No.: 16/976,651

(22) PCT Filed: Feb. 28, 2019
(86) PCT No.: PCT/US2019/020084

§ 371 Date: Aug. 28, 2020

Related U.S. Application Data
(60) Provisional application No. 62/636,566, filed on Feb. 28, 2018.

(51) Int. Cl. A61K 9/51; A61K 9/127

(52) U.S. Cl. CPC A61K 9/5115; A61K 9/127

(57) ABSTRACT

The present disclosure is directed to methods of producing star-like mesoporous silica nanoparticles (SMSNPs) and protocells SMSNPs and their use for targeted drug delivery formulations and systems and for biomedical applications. Also provided are methods of producing monosized protocells from monodisperse SMSNPs and their use for targeted drug delivery formulations and systems and for biomedical applications.

### CROSS-REFERENCE TO RELATED APPLICATIONS

This application claims the benefit of the filing date of U.S. application No. 62/636,566, filed on Feb. 28, 2018, the disclosure of which is incorporated by reference herein.

### BACKGROUND

Mesoporous silica nanoparticles (MSNs) and MSN-supported lipid layer nanoparticles (e.g., bi-layer nanoparticles) are in their modular design and combined properties, including controlled size and shape, large internal surface area, tunable pore and surface chemistry, considerable cargo diversity, high specificity and limited toxicity could allow simultaneous attainment and optimization of needed in vivo characteristics (Lin et al., 2012; Ashley et al., 2011; Ashley et al., 2012; Epler et al., 2012; Cauda et al., 2010; Mackowiak et al., 2013; Wang et al., 2013; Zhang et al., 2014). However, the full potential of these platforms has remained unrealized due to difficulty controlling their physicochemical properties and in vivo stability. This is not a unique problem to MSN based carriers, as the confounding effect of nanoparticle aggregation and poor colloidal stability on a broad range of nanoparticles has been attributed as the source of inaccurate and irreproducible results in complex biological systems (Petros et al., 2010; Lin et al., 2012).

In a non-limiting instance, a ‘protocell’ (Ashley et al., 2011; Ashley et al., 2012; Epler et al., 2012) is a supported lipid bi-layer (SLB) shown to have marked efficacy for targeted delivery of anti-cancer drugs, siRNA, and enzymes in vitro ((Ashley et al., 2011; Ashley et al., 2012; Epler et al., 2012). However, preliminary in vivo experiments conducted in an ex ovo chicken embryo model suggested that these ‘first generation’ protocells rapidly became trapped in the capillary bed and engulfed by immune cells. The synthesis of MSN ‘cores’ by evaporation induced self-assembly (EISA) (Lu et al., 1999), leads to a wide particle size distribution (about 20 to about 800 nm). Subsequent calcinations resulted in irreversible particle aggregation (large hydrodynamic size, >500 nm), a characteristic that was responsible for the impaired circulation times.

### SUMMARY

The present disclosure provides for the synthesis of protocells with control over size, shape, pore structure and/or pore size, while optionally maintaining particle size monodispersity. As described herein, star-like mesoporous silica nanoparticles (SMSNs or SMSNPs) are provided that exhibit ultra large mesopores (e.g., about 25 to 40 nm) while keeping an excellent monodispersity, which previously represented a major problem facing the efforts to create large pore MSNs. In one embodiment, synthesis of the starry monosized MSN was carried out via in situ cooperative thermal-basic etching/rearrangement of dendritic-structure spherical nanoparticles. The advantages of such a system are several, starting from a higher loading capacity of large molecules, and the ability to co-load multiple large biomolecules (e.g., proteins such as BSA or OVA or nucleic acids such as siRNA or mRNA, or combinations thereof). The SMSNs may be employed in biomedical applications as well as in catalysis, e.g., where the SMSNs carry noble metal-based catalysts, separation, e.g., in chromatography, filtration, adsorption, for example, to decontaminate solutions, and in engineering, e.g., by doping construction materials with SMNSs to alter their material properties. In one embodiment, the SMSNs are within a lipid layer, therebyforming a protocell. In one embodiment, a protocell comprises a lipid bilayer and the SMSN. In one embodiment, the lipid layer comprises about 40% to about 50% mol of DOTAP, about 40% to about 50% mol of cholesterol, and optionally about 2% to about 6% mol of DOPE, and optionally about 1% to about 3% mol of DSPE PEG. In one embodiment, the lipid layer comprises about 70% mol to about 80% mol of DSPC, about 10% mol to about 30% mol of cholesterol, and optionally about 3% mol to about 8% mol of DSPE PEG. In one embodiment the lipid layer comprises about 75% mol to about 85% mol DMPC, about 10% mol to about 23% mol DOTAP, and about 1% mol to about 7% mol DSPE PEG2K, e.g., 80:14:6 DMPC:DOTAP:DSPE PEG2K or 78:20:2 DMPC:DOTAP:DSPE PEG2K. In one embodiment the lipid layer comprises about 55% mol to about 75% mol DMPC, about 10% mol to about 23% mol DOTAP, about 3% mol to about 25% mol cholesterol, and about 1% mol to about 7% mol DSPE PEG2K, e.g., 74:15:5:6 or 59:15:20:6 DMPC:DOTAP:chlolesterol:DSPE PEG 2K. In one embodiment the lipid layer comprises about 35% mol to about 50% mol DPPC, about 15% mol to about 30% mol DOTAP, about 25% mol to about 35% mol cholesterol, and about 1% mol to about 75% mol DSPE PEG2K, e.g., 42:25:30:3 DPPC:DOTAP:chlolesterol:DSPE PEG 2K.

In one embodiment, a population of monosized protocells comprising a population of monodisperse star-like mesoporous silica nanoparticles (SMSNPs or SMSNs) is provided, each of said nanoparticles comprising a lipid layer, e.g., a bi-layer or multi-lamellar, coating (e.g., fused thereto), e.g., completely covering the surface of the SMSNPs. In one embodiment said population of protocells exhibits a polydispersity index (Pdl or DPI) of less than about 0.4 to no more than about 0.8. In one embodiment said population of protocells exhibits a polydispersity index of less than about 0.2 to no more than about 0.4. In one embodiment said population of protocells exhibits a polydispersity index of less than about 0.1 to no more than about 0.2.

In one embodiment, a population of monodisperse protocells is provided comprising a population of SMSNPs to each of which is coated with (fused thereto) a lipid bi-layer, said lipid bi-layer completely covering the surface of said SMSNPs, said lipid bi-layer being fused onto said nanoparticles. In one embodiment, at least one lipid in the bilayer at a weight ratio of at least about 200% by weight, e.g., about 200% to about 1000% by weight (e.g., about 2:1 to about 10:1) of said population of nanoparticles, wherein said lipid is at least one cationic, anionic or zwitterionic lipid, e.g., at least one zwitterionic lipid, optionally comprising cholesterol and further optionally comprising a lipid containing a functional group to which may be covalently bonded a targeting or other functional moiety.

Also provided are protocells comprising a population of particle cores comprising monosized SMSNPs and a single lipid bi-layer fused (e.g., a supported lipid bi-layer, SLB) onto the surface of each nanoparticle, said lipid bi-layer comprising at least one lipid and being fused onto said nanoparticle as a monosized liposome in aqueous, e.g., a buffer, solution, wherein said liposome has an internal surface area which is equal to or greater than the external surface area of said nanoparticle. In one embodiment, the lipid bi-layer comprises about 50 to about 99.99 mole percent of at least one anionic, cationic or zwitterionic lipid, e.g., a phospholipid, or at least one zwitterionic phospholipid. In alternative embodiments, the lipid bi-layer comprises 0 to about 50 mole percent, at least about 0.1 up to about 50 mole percent cholesterol (a minor component of cholesterol), for example, about 0.1 to about 10 mole percent, about 0.5 to about 1.5 mole percent, about 1 mole percent cholesterol, about 0.01 to about 25 mole percent, about 0.1 to about 20 mole percent, about 0.25 to about 10 mole percent, or about 0.5 to about 5 to 7.5 mole percent of at least one lipid which contains a functional group to which a targeting moiety (e.g., a peptide, polypeptide such as a monoclonal antibody, etc. or agonist/antagonist of a receptor) or other functional moiety (e.g., a fusogenic peptide or a drug, among numerous others such as toll receptor agonists for immunogenic compositions) may be covalently attached.

In some embodiments, the protocells comprise a SLB which has a lipid transition temperature or Tₘ which is greater than the temperature at which the protocells are stored or used. Accordingly, by utilizing a SLB with a Tₘ which is greater than the temperature at which the protocells are stored or used, the monosized protocells exhibit extended storage stability when stored in an aqueous solution and colloidal stability when these compositions containing these protocells are used to treat patients and subjects.

SMSNPs may range in diameter from about 1 nm to about 500 nm, about 5 nm to about 350 nm, about 10 nm to about 300 nm, about 15 nm to about 250 nm, about 20 nm to about 200 nm, about 25 nm to about 350 nm, or about 20 nm to about 100 nm. In one embodiment, the SMSNPs are about 80 to about 100 nm in diameter. In one embodiment in a population of monodisperse SMSNPs, each SMSNP does not vary more than about 5% from the average diameter of the SMSNPs in the population and exhibits a polydispersity index (Pdl or DPI) of less than about 0.8, or less than about 0.6, e.g., less than about 0.4, less than about 0.3, less than about 0.25 or less than about 0.2.

Monodisperse protocells exhibit colloidal and/or storage stability. In particular, monodisperse protocells exhibit colloidal stability and storage stability in aqueous solution (water, buffer, blood, plasma, etc.) such that the protocells maintain their monodispersity for a period of at least several hours (about 2, 3, 4, 5 or 6 hours), at least about 12 hours, at least about 24 hours, at least about two days, three days, four days, five days, six days, one week, two weeks, four weeks, two months, three months, four months, five months, six months, one year or longer. In one embodiment, the protocells are stored in phosphate buffered saline solutions, saline solution (isotonic saline solution), other aqueous buffer solutions, or water (especially distilled water). The monodispersed protocells maintain their monodispersity in blood, plasma, serum and/or other body fluids for extended periods of time.

Monodisperse protocells may further comprise at least one additional component, for example, a cell targeting species (e.g., a peptide, antibody, such as a monoclonal antibody, an affibody or a small molecule moiety which binds to a cell, among others); a fusogenic peptide that promotes endosomal escape of protocells; a cargo, including one or more drugs (e.g., an anti-cancer agent, anti-viral agent, antibiotic, antifungal agent, etc.); a polynucleotide, such as encapsulated DNA, double stranded linear DNA, a plasmid DNA, small interfering RNA, small hairpin RNA, microRNA, a peptide, polypeptide or protein, an imaging agent, or a mixture thereof, among others), wherein one of said cargo components is optionally conjugated further with a nuclear localization sequence.

In certain embodiments, protocells comprise a nanoporous silica core with a supported lipid bi-layer a cargo comprising at least one therapeutic agent (for example, an anti-viral agent, antibiotic or an anti-cancer agent which optionally facilitates cancer cell death, such as a traditional small molecule, a macromolecular cargo, e.g., siRNA such as S565, S7824 and/or s10234, among others, shRNA or a protein toxin such as a ricin toxin A-chain or diphtheria toxin A-chain) and/or a packaged plasmid DNA (in certain embodiments-histone packaged) disposed within the nanoporous silica core (e.g., supercoiled as otherwise described herein in order to more efficiently package the DNA into protocells as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Protocells may include a targeting peptide which targets cells for therapy (e.g., cancer cells in tissue to be treated, infected cells or other cells requiring therapy) such that binding of the protocell to the targeted cells is specific and enhanced and a fusogenic peptide that promotes endosomal escape of protocells and encapsulated DNA. Protocells may be used in therapy or diagnostics, more specifically to treat cancer and other diseases, including viral infections, including hepatocellular (liver) and other cancers which occur secondary to viral infection. In other aspects, protocells use binding peptides which selectively bind to cancer tissue (MET peptides for example, as disclosed in WO 2012/149376, published Nov. 1, 2012 and CRLF2 peptides, for example, as disclosed in WO 2013/103614, published Jul. 11, 2013, relevant portions of which applications are incorporated by reference herein).

In another embodiment, a storage stable composition is provided comprising a population of monodisperse protocells in an aqueous solution such as buffered saline, water, or isotonic saline solutions, among others.

In an additional embodiment, pharmaceutical compositions (e.g., storage stable compositions) are provided comprising an effective amount of a population of protocells as described herein, in combination with at least one carrier, additive and/or excipient.

In still another embodiment, a method of producing monodisperse protocells is provided. In one embodiment, the method includes providing a population of SMSNPs and exposing said nanoparticles to a population of monosized liposomes comprising at least one lipid (the lipid mixture may be simple or complex, depending on the ultimate function of the protocell), the liposome to SMSNP mass ratio being in one example at least 2:1 and up to about 10:1 (the liposomes may have an internal surface area which is greater than the external surface area of the nanoparticles), wherein the nanoparticles are exposed to the liposomes in an aqueous solution (e.g., an aqueous buffer solution such as phosphate buffered saline solution, although other solutions, including buffered saline solutions may be used). In one embodiment, the monosized liposomes and SMSNPs are combined in buffered saline solution, sonicated or otherwise agitated for several seconds up to a minute or more) to allow the liposomal lipid to coat/fuse to the nanoparticles and the non-fused liposomes in solution are removed/separated from the protocells, for instance, by centrifugation. The pelleted protocells are redispersed at least once (e.g., in phosphate buffered saline solution or other solutions in which the protocells are to be stored and/or used) via agitation (e.g., sonication).

In still another embodiment, therapeutic methods comprise administering a pharmaceutical composition comprising a population of monosized protocells to a patient in need in order to treat a disease state or condition from which the patient is suffering. The disease state includes but is not limited to cancer, a viral infection, a bacterial infection, a fungal infection or other infection.

Thus, the disclosure provides therapeutic formulations with increased therapeutic efficacy in vivo. The dramatic therapeutic efficacy of numerous targeted nanoparticle-based delivery platforms observed in vitro has rarely translated into similar performance in vivo. In exceedingly complex living systems, particle polydispersity, sequestration, and instability have limited the delivery of cargos to specific cell types despite the presence of effective targeting agents. Described herein is a process for the synthesis and characterization of monodisperse mesoporous silica-supported lipid bi-layer nanoparticles (e.g., protocells) designed to exhibit in vivo stability and targeted cell binding. Specific aspects of the modular synthesis protocol allows for precise control of size, shape, pore structure, and surface chemistry that can be tailored to achieve colloidal stability and targeted binding for a range of applications. The demonstrated in vitro stability attributed to the supported lipid bi-layer was confirmed in vivo using real-time, high resolution microscopic analysis in a chicken embryo chorioallantoic membrane (CAM) model combined with hydrodynamic size analysis. Moreover, by establishing synthetic protocols that enabled colloidal stability and avoided non-specific binding of non-targeted protocells, antibody conjugation was demonstrated to direct highly selective binding in vivo.

In one embodiment, one or more of the populations of protocells (often at least two and in certain embodiments all populations of the protocells) comprise a cargo, at least one fusogenic peptide (e.g., R8 octa-arginine to facilitate cellular uptake of the protocells) and at least one targeting species, e.g., to facilitate binding of the protocells to a target on the antigen presenting cells in the lipid bi-layer of the protocell; one or more populations of protocells in said composition (often at least two and in some embodiment all of the protocells) comprise at least one endosomolytic peptide in the lipid bi-layer. One population of protocells comprises at least one viral antigen (which may be a full length viral protein) in the lipid bi-layer or optional aqueous layer of said protocell. This population may comprise an endosomolytic peptide or may exclude such a peptide and one or more populations of protocells in the composition is loaded in the core of said protocell with a viral protein, such as a full length viral protein which is optionally ubiquitinylated (and presented as a fusion protein) and/or a plasmid DNA encoding at least one viral protein (e.g., a full length viral protein), which is optionally and labeled with ubiquitin (expressed as a fusion protein), this protocell population may comprise an endosomolytic peptide. Optionally, one or more populations of protocells in said composition are loaded with at least one bioactive agent, for instance an anti-viral agent.

Pharmaceutical compositions are provided comprising a population of multilamellar or unilamellar protocells in an immunogenic effective amount in combination with at least one additive, excipient and/or carrier. The pharmaceutical composition may comprise additional bioactive agents and other components such as adjuvants (these may also be incorporated into the protocell. Compositions may be used to induce an immunogenic response and/or protective effective against any number of viral infections.

In another embodiment, methods of instilling immunity and/or an immunogenic response or vaccinating a patient or subject at risk for a disease (e.g., an infection such as a viral infection), are provided. The methods include administering a composition to a patient or subject in need in order to induce an immunogenic response in that patient or subject to a virus in order to reduce the likelihood that said patient or subject will become infected with said virus and/or to reduce the likelihood that a virus will cause an acute or chronic infection in said patient or subject.

In one embodiment, a hybrid bilayer protocell is provided comprising a star-like mesoporous silica nanoparticle (SMSNP or SMSN) which is coated on its surface with a hydrocarbon layer, often comprising a silyl hydrocarbon (generally, a C₆-C₄₀ linear, branched or cyclic silylhydrocarbon (e.g., alkylsilane), a C₈-C₃₂ linear, branched or cyclic silylhydrocarbon (e.g., alkyl silane), a C₁₀-C₂₈ linear, branched or cyclic silylhydrocarbon (e.g., alkyl silane or), or a C₁₂-C₂₈ linear, branched or cyclic silyl hydrocarbon (alkyl silane)), the hydrocarbon layer being further coated with a lipid monolayer and a hydrophobic cargo, often a hydrophobic drug loaded into the hybrid bilayer protocell. In alternative embodiments, the hydrocarbon layer comprises a lipid with a primary amine modified headgroup, for example, an amine-containing phospholipid (e.g. DOPE, DMPE, DPPE or DSPE) which is conjugated to the surface of the MSNP through a carboxyl group formed on the surface of the MSNP and a crosslinking agent which crosslinks the surface of the MSNP (through the carboxylic acid moiety) with the amine group of the primary amine containing lipid. The loaded hybrid lipid protocell may be formulated in pharmaceutical dosage form for administering to a patient for the treatment or diagnosis of disease and/or related conditions. In certain embodiments, the hybrid bilayer protocell may contain on the surface of the lipid monolayer PEG groups, targeting peptides and other components which facilitate the administration of the hydrophobic cargo to a particular target, including a cell.

In one embodiment, SMSNPs are synthesized utilizing methods described herein. After formation of the SMSNP, the SMSNP may then be reacted with a chlorosilane hydrocarbon to covalently bond (through Si—O—Si) the silyl hydrocarbon to the surface of the MSNP. The step of reacting the chlorosilane hydrocarbon to the MSNP may occur before or after hydrothermal treatment (e.g., between about 12 and 24 hours at elevated temperatures, e.g. 70° C.).

Alternatively, the SMSNPs may be reacted with a carboxylation agent (e.g., 3-(Triethoxysilyl)propylsuccinic anhydride or other agent to incorporate a carboxyl group on the surface of the MSN) at about 0.1% to about 20% of the molar ratio of TEOS or other silica precursor) for a time sufficient for the carboxylation agent to react with the surface of the MSNP to provide a carboxyl moiety on the surface of the MSNP. The carboxylation step may occur before or after hydrothermal treatment. The carboxylated MSNP is thereafter reacted with a crosslinking agent, e.g., EDC and the crosslinked MSNP is further reacted with an amine containing phospholipid (DOPE, DMPE, DPPE, DSPE or other amine-containing phospholipid to provide a hydrocarbon group on the surface of the MSNP through the crosslinking agent.

The SMSNPs which have hydrocarbon surfaces are then mixed with one or more phospholipids, generally, a mixture of a phospholipid containing a PEG group as otherwise described herein and another phospholipid as described herein. The hydrocarbon coated SMSNPs and phospholipid are mixed in solvent (often chloroform or methylene chloride) often along with a cargo to be incorporated into the final hybrid bilayer protocell and dried together (evaporation of solvent) to form a film. The film is then hydrated with PBS or other buffer and washed several times to form the final MSNPs containing cargo. The cargo may be loaded into the hybrid bilayer protocells at the time that the phospholipid is coated/fused onto the SMSNP or alternatively, the cargo may be added at the time after film formation by incorporating the hydrophobic cargo into the hybrid bilayer protocell when the film is hydrated with buffer.

Hybrid bilayer protocells, in addition to containing at least one hydrophobic cargo, may also include one or more of the following: a targeting species including, for example, targeting peptides including oligopeptides, antibodies, aptamers, and PEG (polyethylene glycol) (including PEG covalently linked to specific targeting species); a cell penetration peptide such as a fusogenic peptide or an endosomolytic peptide as otherwise described herein; a hybrid bilayer protocell comprising a mesoporous silica nanoparticle (SMSNP) with a hydrocarbon coating on said SMSNP and a lipid monolayer coated onto said hydrocarbon coating, wherein said protocell is loaded with a hydrophobic cargo. In one embodiment, the hydrocarbon coating comprises a C₆-C₄₀ siylhydrocarbon. In one embodiment, the hydrocarbon coating comprises is a C₁₂-C₂₈ alkyl silane. In one embodiment, the hydrocarbon coating is formed by reacting a chlorosilylhydrocarbon with the surface of the SMSNP. In one embodiment, the hydrocarbon is formed by reacting carboxylic moieties on the surface of the MSNP with a lipid comprising a primary amine modified headgroup through a crosslinking agent. In one embodiment, the lipid is DOPE, DMPE, DPPE or DSPE. In one embodiment, the crosslinking agent is selected from the group consisting of 1-Ethyl-3-[3-dimethylaminopropyl]carbodiimide hydrochloride (EDC), succinimidyl 4[N-maleimidomethyl]cyclohexane-1-carboxylate (SMCC), Succinimidyl 6-[β-Maleimidopropionamido]hexanoate (SMPH), N-[β-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP). In one embodiment, the lipid monolayer comprises a pegylated phopholipid. In one embodiment, the lipid monolayer comprises a mixture of a phospholipid and a pegylated phospholipid. In one embodiment, the

lipid monolayer comprises DSPE-PEG and/or DOPE-PEG (wherein the PEG average molecular weight is 2000) and optionally one or more of DHPC, DMPC, DOPE, DPPC and cholesterol. In one embodiment, the lipid monolayer includes cholesterol in a minor amount (e.g., less than 50% by weight of the lipid in the lipid monolayer). In one embodiment, the hydrophobic cargo is a drug. In one embodiment, the hydrophobic cargo is a reporter. Also provided is a pharmaceutical composition comprising a population of hybrid protocells in combination with a pharmaceutically acceptable carrier, additive and/or excipient. Further provided is a method of treating a disease state or condition in a patient in need comprising administering to said patient the pharmaceutical composition. In one embodiment, the disease state is cancer.

### BRIEF DESCRIPTION OF THE FIGURES

FIG. 1. TEM images of starry MSNs (SMSNs).

FIG. 2. STEM images of SMSNs.

FIG. 3. DLS of bare and co-loaded SMSN after liposomal fusion. No fusion on unloaded SMSN indicates that the curvature is not suitable for lipid coating. Fusion on (co)-loaded SMSN indicates that loaded proteins fill the pores thus promoting the fusion. There was an increase of size of LC-SMSN after coloading with 2 proteins.

FIG. 4. Porosimetry data for SMSNs. Isotherm shows monolayer adsorption at very low P/P° (inset). Adsorption occurs on high P/P (˜0.8). Absence of a saturation plateau plus the hysterisis form indicate large pores. High BET surface area ˜370 m²/g. Pore Size Distribution clearly shows ultra large pores

with average diameter (25-40 nm) depending on the model.

FIG. 5. Solid state NMR data for SMSNs. There was a relatively moderate condensation degree that promotes fast degradation of the particles.

FIG. 8. SMSNs are obtained under certain conditions.

FIG. 7. SMSNs can be derivatized.

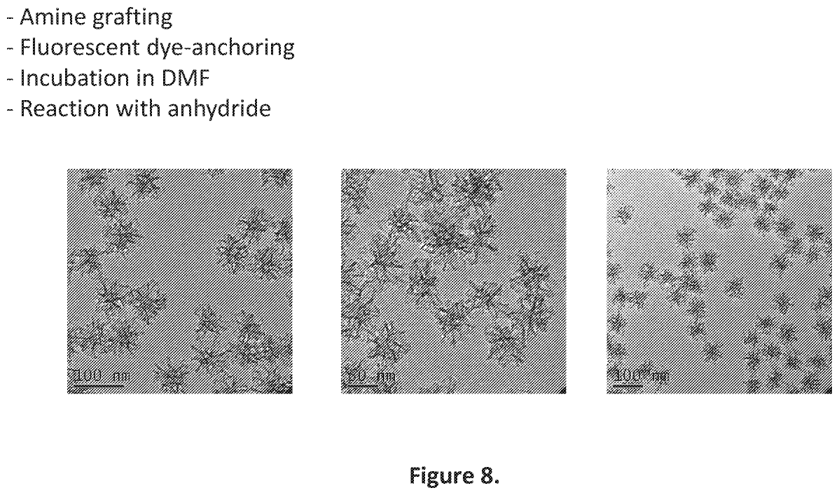

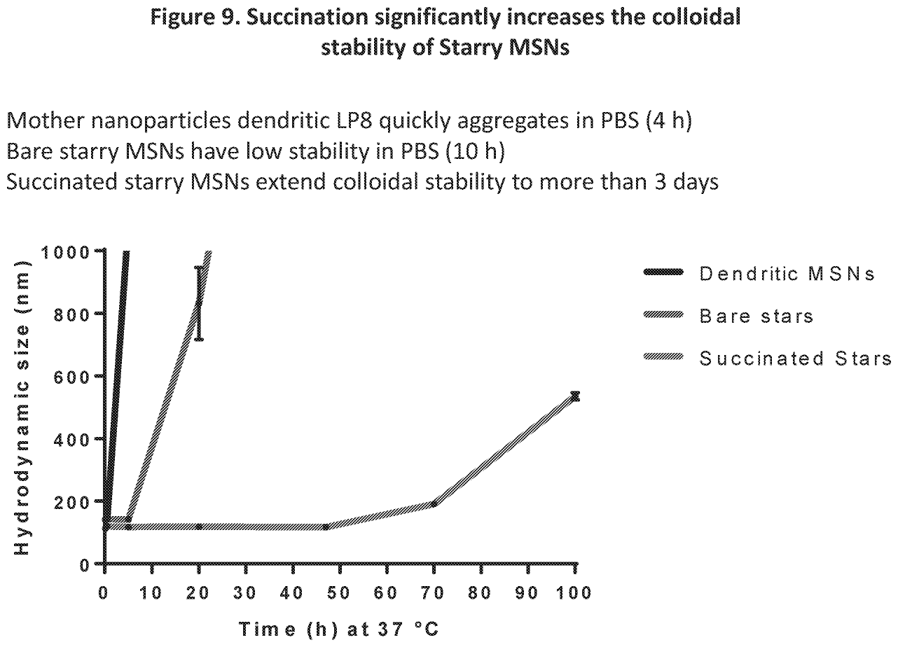

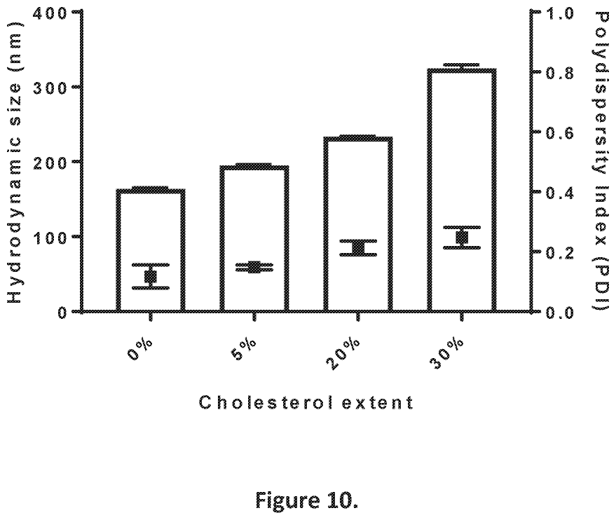

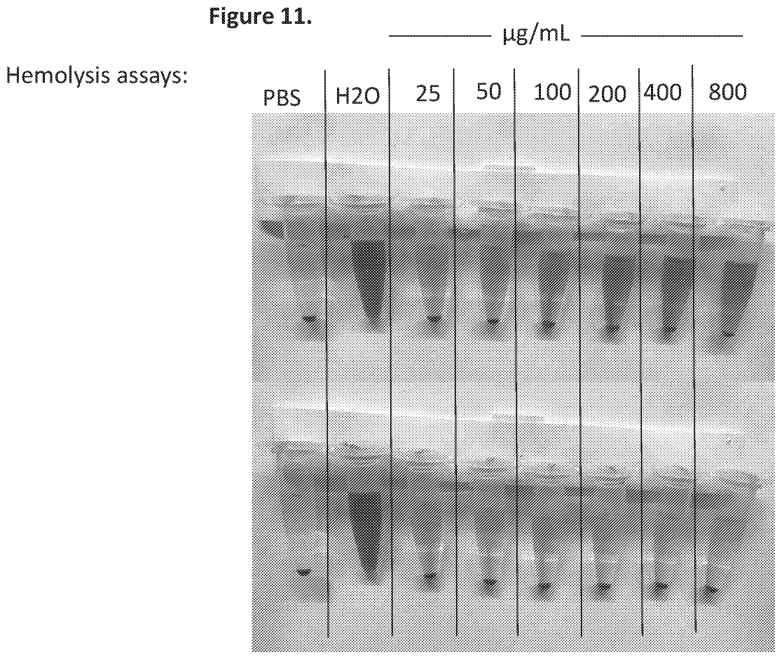

FIG. 8. Starry shape is resistant to organic derivatization.

FIG. 9. Succination significantly increases the colloidal stability of SMSNs. Mother nanoparticles dendritic LP8 quickly aggregate in PBS (4 h). Bare SMSNs have low stability in PBS (10 h). Succinated SMSNs extend colloidal stability to more than 3 days.

FIG. 10. Lipid coating efficacy of SMSNs depends on cholesterol. Cholesterol rigidifies the liposomes and makes their fusion on the rough surface of SMSNs more difficult. This is seen by a size and polydispersity increase while increasing the cholesterol extent within the used liposomal formulations.

FIG. 11. High hemocompatibility of SMSNs. Control (Stöber particles) shows high hemolysis at 50 μg/mL while SMSNs show no or very low hemolysis at 400-800 μg/mL.

### DETAILED DESCRIPTION

These and/or other embodiments of may be readily gleaned from the following description.

### Definitions

Where a range of values is provided, it is understood that each intervening value, to the tenth of the unit of the lower limit unless the context clearly dictates otherwise, between the upper and lower limit of that range and any other stated or intervening value in that stated range is encompassed. The upper and lower limits of these smaller ranges may independently be included in the smaller ranges is also encompassed, subject to any specifically excluded limit in the stated range. Where the stated range includes one or both of the limits, ranges excluding either both of those included limits are also included.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, exemplary methods and materials are now described.

It must be noted that as used herein and in the appended claims, the singular forms “a,” “and” and “the” include plural references unless the context clearly dictates otherwise.

Reference to “about” a value or parameter herein includes (and describes) variations that are directed to that value or parameter per se. For example, description referring to “about X” includes description of “X”.

The term “monodisperse” and “monosized” are used synonymously to describe both mesoporous particles, e.g., nanoparticles (although the particles may range up to about 6 microns in diameter) and protocells (e.g., mesoporous nanoparticles having a fused lipid bi-layer on the surface of the nanoparticles) which are monodisperse.

The term “monosized mesoporous silica nanoparticles” or mMSNPs is used to describe a population of monosized (monodispersed) mesoporous silica nanoparticles. Example particles are produced using a solution-based surfactant directed self-assembly strategy conducted under basic conditions, followed by hydrothermal treatment to provide mMSNPs with tunable core structure, pore sizes and shape. Certain methods for producing silica nanoparticles are described in Lin et al., 2005; Lin et al., 2010; Lin et al., 2011; Chen et al., 2013; Bayu et al., 2009; Wang et al., 2012; Shen et al., 2014; Huang et al., 2011; and Yu et al., 2011, among others. mMSNPs may be provided in various shapes, including star-like, spherical, oval, hexagonal, dendritic, cylindrical, rod-shaped, disc-like, tubular and polyhedral pursuant to the above-described methods. Monodispersity may also be described as having a polydispersity index (Pdl or DPI) of about 0.1 to about 0.3, less than about 0.25, less than about 0.2, or less than about 0.1.

The synthetic procedures for providing monodisperse MSNPs may be varied to vary the contents and size of the mMSNPs, as well as the pore size. In typical synthesis, mMSNPs are produced using a solution based surfactant directed self-assembly strategy conducted under basic conditions (e.g., triethylamine or other weak base), followed by a hydrothermal treatment. Size adjustment may be facilitated by increasing the concentration of catalyst (e.g., ammonium hydroxide). Increasing the concentration of the catalyst will increase the size of the resulting mMSNPs, whereas decreasing the concentration of the catalyst will decrease the size of the resulting mMSNPs. Increasing the amount of silica precursor (e.g., TEOS) will also increase the particle size, as will decreasing the temperature during synthesis. Decreasing the amount of silica precursor and/or increasing the temperature during synthesis will decrease the particle size. All of the above parameters may be modified to adjust the sizes of the mesopores within the nanoparticles. To change the nature of the silica particles, amine-containing silanes such as N-(2-aminoethyl)-3-aminopropyltrimethoxysilane (AEPTMS) or 3-aminopropyttriethoxysilane (APTES) may be added to the solution containing TEOS or other silica precursor. The addition of an amine-containing silane will produce a silica particle with a zeta potential (mV) with a less negative to neutral/positive zeta potential, depending on the amount of amine-containing silane including in the reaction mixture to form the nanoparticles. The nanoparticles have a zeta potential (mV) ranging from about −50 mV to about +35 mV depending upon the amount of amine containing silane added to the synthesis (e.g., from about 0.01% up to about 50% by weight, often about 0.1% to about 20% by weight, about 0.25% to about 15% by weight, about 0.5% to about 10% by weight), with a greater amount of amine containing silane increasing the zeta potential and a lesser amount (to none) providing a nanoparticle with a negative zeta potential.

Surfactants which can be used in the synthesis of mMSNPs include for example, octyltrimethylammonium bromide, decyltrimethylammonium bromide, dodecytrimethylammonium bromide, tetradecytrimethylammonium bromide, benzyldimethylhexadecylammonium chloride, hexadecyltrimethylammonium bromide, hexadecytrimethylammonium chloride, octadecyltrimethylammonium bromide, octadecyltrimethylammonium chloride, dihexadecyldimethylammonium bromide, dimethydioctadecylammonium bromide, dimethylditetradecylammonium bromide, didodecyldimethylammonium bromide, didecyldimethylammonium bromide and didecyldimethylammonium bromide, among others.

The term “protocell” is used to describe a porous nanoparticle surrounded by a lipid bi-layer. In some embodiments, the porous nanoparticle is made of a material comprising silica, polystyrene, alumina, titania, zirconia, or generally metal oxides, organometallates, organosilicates or mixtures thereof.

The term “lipid” is used to describe the components which are used to form lipid bi-layers on the surface of nanoparticles.

Porous nanoparticulates used in protocells include mesoporous silica nanoparticles and core-shell nanoparticles. The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells can be readily obtained using methodologies known in the art. The examples section of the present application provides certain methodology for obtaining protocells. Protocells may be readily prepared, including protocells comprising lipids which are fused to the surface of the silica nanoparticle. See, for example, Liu et al., 2009; Liu et al., 2009: Liu et al., 2009; Lu et al., 1999. Protocells may be prepared according to the procedures which are presented in Ashley et al., 2011; Lu et al., 1999; Caroll et al., 2009, and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, a prism or a whisker. A nanoparticle may include particles having two or more of the aforementioned shapes. In one embodiment, a cross-sectional geometry of the particle may be one or more of circular, ellipsoidal, triangular, toroidal, rectangular or polygonal. In one embodiment, a nanoparticle may consist essentially of non-spherical particles, especially prisms. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles. In one embodiment, a plurality of nanoparticles may consist essentially of hexagonal prism nanoparticles.

The term “monosized protocells” is used to describe a population of monosized (monodisperse) protocells comprising a lipid bi-layer fused onto a mMSNPs as otherwise described herein. In some embodiments, monosized protocells are prepared by fusing the lipids in monosized unilamellar liposomes onto the mMSNPs in aqueous buffer (e.g., phosphate buffered solution) or other solution at about room temperature, although slightly higher and lower temperatures may be used. The unilamellar liposomes which are fused onto the mMSNPs are prepared by sonication and extrusion according to the method of Akbarzadeh et al., 2013 and are monodisperse with hydrodynamic diameters of, in one example, less than about 100 nm, often about 65-95 nm, most often about 90-95 nm, although unilamellar liposomes which can be used may fall outside this range depending on the size of the mMSNPs to which lipids are to be fused and low PDI values (generally, less than about 0.5, e.g., less than 0.2). The mass ratio of liposomes to mMSNPs used to create monosized protocells which have a single lipid bi-layer completely surrounding the mMSNPs is that amount sufficient to provide a liposome interior surface area which equals or exceeds the exterior surface area of the mMSNPs to which the lipid is to be fused. This often is provided in a mass ratio of liposomes to mMSNPs of at least about 2:1, often up to about 10:1 or more, with a range often used being about 2:1 to about 5:1. The resulting protocells are monosized (monodisperse). Monosized protocells may exhibit extended storage stability in aqueous solution, e.g., providing a SLB on the protocell which has a transition temperature Tₘ which is greater than the storage, experimental or administration/therapeutic conditions under which the protocells are stored and/or used. Often the protocell is at least about 25-30 nm in diameter larger than the diameter of the mMSNPs.

The phrase “effective average particle size” as used herein to describe a multiparticulate (e.g., a porous nanoparticulate) means that all particles therein are of an average diameter or within ±5% of the average diameter. In certain embodiments, nanoparticulates have an effective average particle size (diameter) of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, less than about 50 nm, less than about 35 nm, less than about 25 nm, as measured by light-scattering methods, microscopy, or other appropriate methods. In exemplary aspects, the average diameter of SMSNPs ranges from about 75 nm to about 150 nm, often about 75 to about 130 nm, often about 75 nm to about 100 nm.

The term “patient” or “subject” is used throughout the specification within context to describe an animal, generally a mammal, especially including a domesticated animal and for example a human, to whom treatment, including prophylactic treatment (prophylaxis), with the compounds or compositions is provided. For treatment of those infections, conditions or disease states which are specific for a specific animal such as a human patient, the term patient refers to that specific animal. In most instances, the patient or subject is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment. Exemplary bioactive agents include the compounds which are used to treat cancer or a disease state or condition which occurs secondary to cancer and may include anti-viral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease state or condition, including improvement in the disease state or condition through lessening, inhibition, suppression or elimination of at least one symptom, delay in progression of the disease, prevention, delay in or inhibition of the likelihood of the onset of the disease state and/or condition, etc. In the case of microbial infections, these terms also apply to microbial (e.g., viral or bacterial) infections and may include, in certain particularly favorable embodiments the eradication or elimination (as provided by limits of diagnostics) of the microbe (e.g., a virus or a bacterium) which is the causative agent of the infection.

Treatment, as used herein, encompasses both prophylactic and therapeutic treatment, e.g., of cancer (including inhibiting metastasis or recurrence of a cancer in remission), but also of other disease states, including microbial infections such as bacterial, fungal, protest, aechaea, and viral infections, especially including HBV and/or HCV. Compounds can, for example, be administered prophylactically to a mammal in advance of the occurrence of disease to reduce the likelihood of that disease. Prophylactic administration, e.g., a vaccine, is effective to reduce or decrease the likelihood of the subsequent occurrence of disease in the mammal, or decrease the severity of disease (inhibition) that subsequently occurs, especially including metastasis of cancer. Alternatively, compounds can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections. In another embodiment of therapeutic administration, administration of the present compounds is effective to decrease the likelihood of infection or re-infection by a microbe and/or to decrease the symptom(s) or severity of an infection.

The term “prophylactic administration” refers to any action in advance of the occurrence of disease to reduce the likelihood of that disease or any action to reduce the likelihood of the subsequent occurrence of disease in the subject. Compositions can, for example, be administered prophylactically to a mammal in advance of the occurrence of disease to enhance an immunogenic effect and/or reduce the likelihood of that disease, generally a viral disease. Prophylactic administration is effective to reduce or decrease the likelihood of the subsequent occurrence of disease in the mammal, or decrease the severity of disease (inhibition) that subsequently occurs, especially including a microbial (e.g., a viral or bacterial) infection and/or cancer, its metastasis or recurrence.

The term “targeting active species” is used to describe a compound or moiety which is complexed or covalently bonded to the surface of a protocell which binds to a moiety on the surface of a cell to be targeted so that the protocell may selectively bind to the surface of the targeted cell and deposit its contents into the cell. In one embodiment, the targeting active species is a “targeting peptide” including a polypeptide including an antibody or antibody fragment, an aptamer, or a carbohydrate, among other species which bind to a targeted cell. A targeting active species may be peptide of a particular sequence which binds to a receptor or other polypeptide in cancer cells and allows the targeting of protocells to particular cells which express a peptide (be it a receptor or other functional polypeptide) to which the targeting peptide binds. Exemplary targeting peptides include, for example, SP94 free peptide (H₂N-SFSIILTPILPL-COOH, SEQ ID NO: 3), SP94 peptide modified with a C-terminal cysteine for conjugation with a crosslinking agent (H₂N-GLFHAIAHFIHGGWHGLIHGWYGGC-COOH (SEQ ID. NO:4) or an 8 mer polyarginine (H₂N—RRRRRRRR-COOH, SEQ ID NO:5), a modified SP94 peptide (H₂N-SFSIILTPILPLEEEGGC-COOH, SEQ ID NO:6) or a MET binding peptide or CRLF2 binding peptide as disclosed in WO 2012/149376, published Nov. 1, 2012 and CRLF2 peptides, for example as disclosed in WO 2013/103614, published Jul. 11, 2013, relevant portions of which applications are incorporated by reference herein. Other targeting peptides are known in the art. Targeting peptides may be complexed or covalently linked to the lipid bi-layer through use of a crosslinking agent as otherwise described herein.

The term “MET binding peptide” or “MET receptor binding peptide” is used to describe any peptide that binds the MET receptor. These peptides are particularly useful as targeting ligands for cell-specific therapeutics. The following five 7 mer peptide sequences show substantial binding to MET receptor and may be useful as targeting peptides for use on protocells.

(SEQ ID NO: 7)
ASVHFPP (Ala-Ser-Val-His-Phe-Pro-Pro)
(SEQ ID NO: 8)
TATFWFQ (Thr-Ala-Thr-Phe-Trp-Phe-Gln)
(SEQ ID NO: 9)
TSPVALL (Thr-Ser-Pro-Val-Ala-Leu-Leu)
(SEQ ID NO: 10)
IPLKVHP (Ile-Pro-Leu-Lys-Val-His-Pro)
(SEQ ID NO: 11)
WPRLTNM (Trp-Pro-Arg-Leu-Thr-Asn-Met)

Each of these peptides may be used alone or in combination with other MET peptides within the above group or with other targeting peptides which may assist in binding protocells n to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “fusogenic peptide” and “endosomolytic peptide” are used synonymously to describe a peptide which is optionally crosslinked onto the lipid bi-layer surface of the protocells. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative fusogenic peptides for use in protocells include but are not limited to H5WYG peptide, H₂N-GLFHAIAHFIHGGWHGLIHGWYGGC-COOH (SEQ ID. NO:12) or an 8 mer polyarginine (H₂N—RRRRRRRR-COOH, SEQ ID NO:13), among others known in the art. Additional fusogenic peptides include RALA peptide (NH₂-WEARLARALARALARHLARALARALRAGEA-COOH, SEQ ID NO: 14), KALA peptide (NH₂-WEAKLAKALAKALAKHLAKALAKALKAGEA-COOH), SEQ ID. NO:15), GALA (NH₂-WEAALAEALAEALAEHLAEALAEALEALAA-COOH, SEQ ID NO:16) and INF7 (NH2-GLFEAIEGFIENGWEGMIDGWYG-COOH, SEQ ID. NO:17), among others.

Thus, the terms “cell penetration peptide,” “fusogenic peptide” and “endosomolytic peptide” are used to describe a peptide which aids protocell translocation across a lipid bi-layer, such as a cellular membrane or endosome lipid bi-layer and is optionally crosslinked onto a lipid bi-layer surface of the protocells. Endosomolytic peptides are a sub-species of fusogenic peptides as described herein. In both the multilamellar and single layer protocell embodiments, the non-endosomoytic fusogenic peptides (e.g., electrostatic cell penetrating peptide such as R8 octaarginine) are incorporated onto the protocells at the surface of the protocell in order to facilitate the introduction of protocells into targeted cells (APCs) to effect an intended result (to instill an immunogenic and/or therapeutic response as described herein). The endosomolytic peptides (often referred to in the art as a subset of fusogenic peptides) may be incorporated in the surface lipid bi-layer of the protocell or in a lipid sublayer of the multilamellar protocell in order to facilitate or assist in the escape of the protocell from endosomal bodies. Representative electrostatic cell penetration (fusogenic) peptides for use in protocells include an 8 mer poyarginine (H₂N-RRRRRRRR-COOH, SEQ ID NO:1), among others known in the art, which are included in protocells in order to enhance the penetration of the protocell into cells. Representative endosomolytic fusogenic peptides (“endosomolytic peptides) include H₅WYG peptide, H₂N-GLFHAIAHFIHGGWHGLIHGWYGGC-COOH (SEQ ID. NO: 2), RALA peptide (NH₂-WEARLARALARALARHLARALARALRAGEA-COOH, SEQ ID NO: 18), KALA peptide (NH₂-WEAKLAKALAKALAKHLAKALAKALKAGEA-COOH), SEQ ID. NO:19), GALA (NH2-WEAALAEALAEALAEHLAEALAEALEALAA-COOH, SEQ ID NO:20) and INF7 (NH2-GLFEAIEGFIENGWEGMIDGWYG-COOH, SEQ ID. NO:21), among others. At least one endosomolytic peptide is included in protocells in combination with a viral antigen (often pre-ubiquitinylated) and/or a viral plasmid (which expresses viral protein or antigen) in order to produce CD8+ cytotoxic T cells pursuant to a MHC class I pathway.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides and other functional moieties (for example toll receptor agonists for immunogenic) to the phospholipid bi-layer, to link nuclear localization sequences to histone proteins for packaging supercoiled plasmid DNA and in certain instances, to crosslink lipids in the lipid bi-layer of the protocells. There are a large number of crosslinking agents which may be used in many commercially available or available in the literature. Exemplary crosslinking agents for use, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[β-Maleimidopropionic acid] hydrazide (BMPH), NHS-(PEG)ₙ-maleimide, succinimidyl-[(N-maleimidopropionamido)-tetracosaethyleneglycol]ester (SM(PEG)₂₄), and succinimidyl 6-[3′-(2-pyridyldithio)-propionamido] hexanoate (LC-SPDP), among others.

The term “crosslinking agent” is used to describe a bifunctional compound of varying length containing two different functional groups which may be used to covalently link various components to each other. Crosslinking agents may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides to the phospholipid bi-layer, to link nuclear localization sequences to histone proteins for packaging supercoiled plasmid DNA and in certain instances, to crosslink lipids in the lipid bi-layer of the protocells. There are a large number of crosslinking agents which may be used, many commercially available or available in the literature. Exemplary crosslinking agents for use include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[β-Maleimidopropionamido]hexanoate (SMPH), N-[β-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

The term “anti-viral agent” is used to describe a bioactive agent/drug which inhibits the growth and/or elaboration of a virus, including mutant strains such as drug resistant viral strains. In one embodiment, anti-viral agents include anti-HIV agents, anti-HBV agents and anti-HCV agents. In certain aspects, especially where the treatment of hepatocellular cancer is an object of cotherapy, the inclusion of an anti-hepatitis C agent or anti-hepatitis B agent may be combined with other traditional anticancer agents to effect therapy, given that hepatitis B virus (HBV) and/or hepatitis C virus (HCV) is often found as a primary or secondary infection or disease state associated with hepatocellular cancer. Anti-HBV agents which may be used, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use include such agents as boceprevir, daclatasvir, asunapavir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “targeting active species” is used to describe a compound or moiety which binds to a moiety on the surface of a targeted cell so that the protocell may selectively bind to the surface of the targeted cell and deposit its contents into the cell. The targeting active species for use may be a targeting peptide as otherwise described herein, a polypeptide including an antibody or antibody fragment, an aptamer, or a carbohydrate, among other species which bind to a targeted cell, especially an antigen presenting cell.

The term “toll-like receptor (TLR) agonist” includes but is not limited Pam3Cys, HMGB1, Porins, HSP, GLP (agonists for TLR1/2); BCG-CWS, HP-NAP, Zymosan, MALP2, PSK (agonists for TLR 2/6); dsRNA, Poly AU, Poly ICLC, Poly I:C (agonists for TLR 3); LPS, EDA, HSP, Fibrinogen, Monophosphoryl Lipid A (MPLA) (agonists for TLR 4); Flagellin (agonist for TLR 5); Imiquimod (agonist for TLR 7); and ssRNA, PolyG10 and CpG (agonists for TLR 8), as described by Kaczanowka et al., 2013. TLR agonists may be covalently linked to components of the lipid bi-layer using conventional chemistry as described herein above for the fusogenic peptides.

The term “ubiquitin” or “ubiquitinylation” is used throughout the present specification to refer to the use of a ubiquitin protein in combination with a viral antigen (e.g., a full length viral protein) as a fusion protein or conjugated via an isopeptide bond. Ubiquitylation of viral proteins generally speeds the development of immunogenicity. Ubiquitin, also referred to as ubiquitous immunopoietic polypeptide, is a protein involved in ubiquitination in the cell and, facilitates the immunogenic response raised after the protocells are introduced into antigen presenting cells (APCs) by facilitating/regulating the degradation of proteins (via the proteasome and lysosome), coordinating the cellular localization of proteins, activating and inactivating proteins and modulating protein-protein interactions, resulting in an enhancement in antigen processing in both professional and non-professional APCs through exogenous and endogenous pathways.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions, alone or in combination with another agent. It is noted that prophylaxis will rarely be 100% effective; consequently the terms prevention and reducing the likelihood are used to denote the fact that within a given population of patients or subjects, administration with compounds will reduce the likelihood or inhibit a particular condition or disease state (in particular, the worsening of a disease state such as the growth or metastasis of cancer) or other accepted indicators of disease progression from occurring.

“Amine-containing silanes” include, but are not limited to, a primary amine, a secondary amine or a tertiary amine functionalized with a silicon atom, and may be a monoamine or a polyamine such as diamine. For example, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used to modify the SMSNPs.

The term “reporter” is used to describe an imaging agent or moiety which is incorporated into the phospholipid bi-layer or cargo of protocells according to an embodiment and provides a signal which can be measured. The moiety may provide a fluorescent signal or may be a radioisotope which allows radiation detection, among others. Exemplary fluorescent labels for use in protocells (e.g., via conjugation or adsorption to the lipid bi-layer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CelTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of cancer (cancer tissue) in a patient and or the progress of therapy in a patient or subject.

The term “histone-packaged supercoiled plasmid DNA” is used to describe an exemplary component of protocells, which utilize an exemplary plasmid DNA which has been “supercoiled” (e.g., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (e.g., complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments which include a MET binding peptide complexed to the protocell.

The terms “coadminister” and “coadministration” are used synonymously to describe the administration of at least one of the protocell compositions in combination with at least one other agent, often at least one additional anti-cancer agent (as otherwise described herein), which are specifically disclosed herein in amounts or at concentrations which would be considered to be effective amounts at or about the same time. While it is envisioned that coadministered compositions/agents be administered at the same time, agents may be administered at times such that effective concentrations of both (or more) compositions/agents appear in the patient at the same time for at least a brief period of time. Alternatively, in certain aspects, it may be possible to have each coadministered composition/agent exhibit its inhibitory effect at different times in the patient, with the ultimate result being the inhibition and treatment of cancer, especially including hepatocellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising protocells and optionally, to treat any type of cancer, in particular hepatocellular or cervical cancer, among numerous others. Anti-cancer compounds which can be formulated with compounds include, for example, Exemplary anti-cancer agents which may be used include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-10, BIO 140, CC 8490, cilengitide, gimatecan, IL13-PE38QQR, INO 1001, IPdR₁ KRX-0402, lucanthone, LY 317615, neuradiab, vitespen, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrozole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate salt of [D-Ser(But)6,Azgly10] (pyro-Glu-His-Trp-Ser-Tyr-D-Ser(But)-Leu-Arg-Pro-Azgly-NH₂ acetate [C₅₉H₈₄N₁₈O₁₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714, TAK-165, HKI-272, erotinib, lapatinib, canertinib, ABX-EGF antibody, erbitux, EKB-569, PKI-166, GW-572016, lonafamib, BMS-214662, tipifamib, amifostine, NVP-LAQ824, suberoyl anilide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, ratitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deoxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, intereukin-12, IM862, angiostatin, vitaxin, droloxifene, idoxifene, spironolactone, finasteride, cimetidine, trastuzumab, denileukin diftitox, gefitinib, bortezomib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40--(2-hydroxyethyl)-rapamycin, temsiroimus, AP-23573, RAD001, ABT-578, BC-210, LY294002, LY292223, LY292696, LY293684, LY293646, wortmannin, ZM336372, L-779,450, PEG-fllgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, intereukin-11, dexrazoxane, alemtuzumab, all-transretinoic acid, ketoconazole, intereukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, etidronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anti-cancer agent which may be used to inhibit, treat or reduce the likelihood of hepatocellular cancer, or the metastasis of that cancer. Anti-cancer agents which may find use include for example, nexavar (sorafenib), sunitinib, bevacizumab, tarceva (erlotinib), tykerb (lapatinib) and mixtures thereof. In addition, other anti-cancer agents may also be used, where such agents are found to inhibit metastasis of cancer, in particular, hepatocellular cancer.

The term “anti(HCV)-viral agent” is used to describe a bioactive agent/drug which inhibits the growth and/or elaboration of a virus, including mutant strains such as drug resistant viral strains. Exemplary anti-viral agents include anti-HIV agents, anti-HBV agents and anti-HCV agents. In certain aspects, especially where the treatment of hepatocellular cancer is the object of therapy, the inclusion of an anti-hepatitis C agent or anti-hepatitis B agent may be combined with other traditional anti-cancer agents to effect therapy, given that hepatitis B virus (HBV) and/or hepatitis C virus (HCV) is often found as a primary or secondary infection or disease state associated with hepatocellular cancer. Anti-HBV agents which may be used, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtorcitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in include such agents as boceprevir, daclatasvir, asunaprevir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “anti-HIV agent” refers to a compound which inhibits the growth and/or elaboration of HIV virus (I and/or II) or a mutant strain thereof. Exemplary anti-HIV agents for use which can be included as cargo in protocells include, for example, including nucleoside reverse transcriptase inhibitors (NRTI), other non-nucleoside reverse transcriptase inhibitors (e.g., those which are not representative), protease inhibitors, fusion inhibitors, among others, exemplary compounds of which may include, for example, 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof

### Exemplary Monosized Nanostructures

In an embodiment, the nanostructures include a starry mesoporous silica core-shell structure which comprises a porous particle core surrounded by a shell of lipid such as a bi-layer, but possibly a monolayer or multi-layer. The porous silica particle core includes, for example, a porous nanoparticle surrounded by a lipid bi-layer. In some non-limiting instances, these lipid bi-layer surrounded nanostructures are referred to as “protocells” or “functional protocells” and have a supported lipid bi-layer membrane structure. However, the porous nanoparticle may be surrounded by other naturally occurring or synthetic polymers and those may also be referred to as “protocells.” In some embodiments, the porous particle core of the protocells can be loaded with various desired species (“cargo”), including small molecules (e.g., anti-cancer agents as otherwise described herein), large molecules (e.g., including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others) or a reporter polypeptide (e.g., fluorescent green protein, among others) or semiconductor quantum dots or combinations thereof. In certain exemplary aspects, the protocells are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-cancer agents, anti-viral agents and antibiotics, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In some embodiments, the lipid bi-layer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

In some embodiments, the protocells particle size distribution is monodisperse. In certain embodiments, protocells generally range in size from greater than about 8-10 nm to about 5 μm in diameter, e.g., about 20-nm-3 μm in diameter, about 10 nm to about 500 nm, about 20-200-nm (including about 150 nm, which may be a mean or median diameter), about 50 nm to about 150 nm, about 75 to about 130 nm, or about 75 to about 100 nm. As discussed above, the protocell population is considered monodisperse based upon the mean or median diameter of the population of protocells. Size is very important to therapeutic and diagnostic aspects as particles smaller than about 8-nm diameter are excreted through kidneys, and those particles larger than about 200 nm are often trapped by the liver and spleen. Thus, an embodiment on smaller monosized protocells are provided of less than about 150 nm for drug delivery and diagnostics in the patient or subject.

In certain embodiments, protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Exemplary pore sizes of mesopores range from about 2-nm; they can be monosized or bimodal or graded—they can be ordered or disordered (essentially randomly disposed or worm-like).

Mesopores (IUPAC definition 2-50-nm in diameter) may be ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g., if a templating moiety in the aerosol process is not used. They could also be enlarged to macropores, e.g., 50-nm in diameter.

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargos through these attractive interactions.

In certain embodiments, the surface area of nanoparticles, as measured by the N2 BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM of cargo per 10¹⁰ particles where values often ranging from 2000-100 μM per 10¹⁰ particles are used. Exemplary protocells exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the protocells according to one embodiment, the surface area is mainly internal as opposed to the external geometric surface area of the nanoparticle.

The lipid bi-layer supported on the porous particle according to one embodiment has a lower melting transition temperature, e.g., is more fluid than a lipid bi-layer supported on a non-porous support or the lipid bi-layer in a liposome. This is sometimes important in achieving high affinity binding of targeting ligands at low peptide densities, as it is the bi-layer fluidity that allows lateral diffusion and recruitment of peptides by target cell surface receptors. One embodiment provides for peptides to cluster, which facilitates binding to a complementary target.

The lipid bi-layer may vary significantly in composition. Ordinarily, any lipid or polymer which is may be used in liposomes may also be used in protocells. Exemplary lipids are as otherwise described herein. Particular lipid bi-layers for use in protocells comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bi-layer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment are that they are stable at pH 7, e.g., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Protocells may exhibit at least one or more a number of characteristics (depending upon the embodiment) which distinguish them from prior art protocells: 1) In contrast to the prior art, an embodiment specifies monosized nanoparticles whose average size (diameter) is less than about 200-nm—this size is engineered to enable efficient cellular uptake by receptor mediated endocytosis and to minimize binding and uptake by non-target cells and organs; 2) Monodisperse sizes to enable control of biodistribution of the protocells; 3) To targeted nanoparticles that bind selected to cells based upon the inclusion of a targeting species on the protocell; 4) To targeted nanoparticles that induce receptor mediated endocytosis; 5) Induces dispersion of cargo into cytoplasm of targeted cells through the inclusion of fusogenic or endosomolytic peptides; 6) Provides particles with pH triggered release of cargo; 7) Exhibits controlled time dependent release of cargo (via extent of thermally induced crosslinking of silica nanoparticle matrix); 8) Exhibit time dependent pH triggered release; 9) Contain and provide cellular delivery of complex multiple cargoes; 10) Cytotoxicity of target cancer cells; 11) Diagnosis of target cancer cells; 12) Selective entry of target cells; 13) Selective exclusion from off-target cells (selectivity); 14) Enhanced fluidity of the supported lipid bi-layer; 15) Sub-nanomolar and controlled binding affinity to target cells; 16) Sub-nanomolar binding affinity with targeting ligand densities; and/or 17) Colloidal and storage stability of compositions comprising protocells.

Various embodiments provide nanostructures which are constructed from nanoparticles which support a lipid bi-layer(s). In some embodiments, the nanostructures include, for example, a core-shell structure including a porous particle core surrounded by a shell of lipid bi-layer(s). The nanostructure, e.g., a porous silica nanostructure as described above, supports the lipid bi-layer membrane structure.

In some embodiments, the lipid bi-layer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bi-layers, can vary widely in molecular weight (although PEG ranging from about 10 to about 100 units of ethylene glycol, about 15 to about 50 units, about 40 to 50 units, about 15 to about units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bi-layer.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the given the fact that cholesterol may be an important component of the lipid bi-layer of protocells according to an embodiment. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

Core-shell nanoparticles comprise a core and shell. The core, in one embodiment, comprises silica and an absorber molecule. The absorber molecule is incorporated in to the silica network via a covalent bond or bonds between the molecule and silica network. The shell comprises silica.

In one embodiment, the core may be independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer see also Pluedemann, 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosures of which are incorporated herein by reference.

In certain embodiments of a protocell, the lipid bi-layer is comprised of one or more lipids selected from the group consisting of phosphatidyl-cholines (PCs) and cholesterol.

In certain embodiments, the lipid bi-layer is comprised of one or more phosphatidyl-cholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

In other embodiments: (a) the lipid bi-layer is comprised of a mixture of (1) DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid bi-layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidycholine (DMPC), and dimyristoylphosphatidylglycerol (DMPG).

In still other embodiments, the lipid bi-layer comprises one or more PEG-containing phospholipids, for example 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3-phosphoethanolamine-N-[amino(polyethylene glycol)] (DSPE-PEG-NH₂) (DSPE-PEG). In the PEG-containing phospholipid, the PEG group ranges from about 2 to about 250 ethylene glycol units, about to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N-[amino(polyethylene glycol)-2000] (DSPE-PEG₂₀₀₀-NH₂) which can be used to covalent bind a functional moiety to the lipid bi-layer.

In one illustrative embodiment of a protocell: (a) the one or more pharmaceutically-active agent, which for example include at least one anti-cancer agent; (b) less than around 10% to around 20% of the pharmaceutically-active agent is released from the porous nanoparticulates in the absence of a reactive oxygen species; and (c) upon disruption of the lipid bi-layer as a result of contact with a reactive oxygen species, the porous nanoparticulates release an amount of the agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anti-cancer agent that would have been released had the lipid bi-layer been lysed with 5% (w/v) Triton X-100.

One illustrative embodiment of a protocell comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; (b) are loaded with a siRNA or ricin toxin A-chain; and (c) that are encapsulated by and that support a lipid bi-layer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bi-layer comprises a cationic lipid and one or more zwitterionic phospholipids.

Monosized protocells can comprise a wide variety of pharmaceutically-active ingredients such as nucleic acid, e.g., DNA.

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced pursuant to the teachings herein. The DNA may also be double stranded linear DNA, instead of plasmid DNA, which also may be optionally supercoiled and/or packaged with histones or other packaging components. Other histone proteins which may be used in this aspect include, for example, H1F, H1F0, H1FNT, H1FOO, H1FX, H1H1, HIST1H1A, HIST1H1B, HIST1H1C, HIST1H1D, HIST1H1E, HIST1H1T, H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H31, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H41, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR[PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998, TIBS, 23, 185-9 (1998); and Murat Cokol et al., “Finding nuclear localization signals”, at the website ubic.bioc.columbia.edu/papers/2000 nls/paper.html#tab2.

In general, protocells are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bi-layer(s) as generally described herein.

The porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In some aspects, the lipid bi-layer is fused onto the porous particle core to form the monosized protocells. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bi-layer which is used to prepare protocells are monosized liposomes which can be prepared, for example, by extrusion of liposomes prepared by bath sonication through a filter with pore size of, for example, about 100 nm, using standard protocols known in the art or as otherwise described herein. Alternatively, the monosized liposomes are prepared from lipids using bath and probe sonication without extrusion. While the majority of the monosized liposomes are unilamellar when prepared using extrusion, in the absence of extrusion, the monosized liposomes will have an appreciable percent of multilamellar liposomes. The monosized liposomes can then be fused with the porous particle cores, for example, by sonicating (e.g., bath sonication, other) a mixtures of monosized liposomes and SMSNPs in buffered saline solution (e.g., PBS), followed by separation (centrifugation) and redispersing the pelleted protocells via sonication in a saline or other solution. In exemplary embodiments, excess amount of liposome (e.g., at least twice the amount of liposome to SMSNP) is used. To improve the protocell colloidal and/or storage stability of the protocell composition, the transition melting temperature (Tₘ) of the lipid bi-layer should be greater than the temperature at which the protocells are to be stored and/or used. For storage stable liposomes, the inclusion of appreciable amounts of saturated phospholipids in the lipid bi-layer is often used to increase the Tₘ of the lipid bi-layer.

In certain diagnostic embodiments, various dyes or fluorescent (reporter) molecules can be included in the protocell cargo (as expressed by as plasmid DNA) or attached to the porous particle core and/or the lipid bi-layer for diagnostic purposes. For example, the porous particle core can be a silica core or the lipid bi-layer and can be covalently labeled with FITC (green fluorescence), while the lipid bi-layer or the particle core can be covalently labeled with FITC Texas red (red fluorescence). The porous particle core, the lipid bi-layer and the formed protocell can then be observed by, for example, confocal fluorescence for use in diagnostic applications. In addition, as discussed herein, plasmid DNA can be used as cargo in protocells, such that the plasmid may express one or more fluorescent proteins such as fluorescent green protein or fluorescent red protein which may be used in diagnostic applications.

In various embodiments, the protocell is used in a synergistic system where the lipid bi-layer fusion or liposome fusion (i.e., on the porous particle core) is loaded and sealed with various cargo components with the pores (e.g., mesopores) of the particle core, thus creating a loaded protocell useful for cargo delivery across the cell membrane of the lipid bi-layer or through dissolution of the porous nanoparticle, if applicable. In certain embodiments, in addition to fusing a single lipid (e.g., phospholipids) bi-layer, multiple bi-layers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., anti-cancer drugs and/or anti-viral drugs such as anti-HBV or anti-HCV drugs), peptides, proteins, antibodies, DNA (especially plasmid DNA, including the exemplary histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In some embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from, for example, about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bi-layer is fused onto the silica surface showing a fusion and synergistic loading mechanism. In this manner, fusion of a non-negatively charged (i.e., positively charged or neutral) lipid bi-layer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA for example modified with a nuclear localization sequence can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs, bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded protocells can deliver their cargo into targeted cells for therapy or diagnostics.

In various embodiments, the protocells and/or the loaded protocells can provide a targeted delivery methodology for selectively delivering the protocells or the cargo components to targeted cells (e.g., cancer cells). For example, a surface of the lipid bi-layer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a MET binding peptide as otherwise described herein.

For example, by providing a targeting active species (e.g., a targeting peptide) on the surface of the loaded protocell, the protocell selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bi-layer, a large number of the cargo-loaded protocells can be recognized and internalized by this specific cancer cells due to the specific targeting of the exemplary SP94 or a MET or a CRLF2 binding peptide with the cancer (including liver) cells. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bi-layer on the porous particle core, the cargo components can be released from the pores of the lipid bi-layer, transported across the protocell membrane of the lipid bi-layer and delivered within the targeted cell. In embodiments, the release profile of cargo components in protocells can be more controllable as compared with when only using liposomes as known in the prior art. The cargo release can be determined by, for example, interactions between the porous core and the lipid bi-layer and/or other parameters such as pH value of the system. For example, the release of cargo can be achieved through the lipid bi-layer, through dissolution of the porous silica; while the release of the cargo from the protocells can be pH-dependent.

In certain embodiments, the pH value for cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

In certain embodiments, the inclusion of surfactants can be provided to rapidly rupture the lipid bi-layer, transporting the cargo components across the lipid bi-layer of the protocell as well as the targeted cell. In certain embodiments, the phospholipid bi-layer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bi-layer. One example would be gold or magnetic nanoparticles that could use light or heat to generate heat thereby rupturing the bi-layer. Additionally, the bi-layer can be tuned to rupture in the presence of discrete biophysical phenomena, such as during inflammation in response to increased reactive oxygen species production. In certain embodiments, the rupture of the lipid bi-layer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bi-layers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid bi-layer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bi-layer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Pharmaceutical compositions may comprise an effective population of protocells as otherwise described herein formulated to effect an intended result (e.g., therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions may also comprise an addition bioactive agent or drug, such as an anti-cancer agent or an anti-viral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 95%, about 0.25% to about 85%, about 0.5% to about 75% by weight of a compound/composition or compounds/compositions, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or would be apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject.

Methods of treating patients or subjects in need for a particular disease state or infection (including cancer and) comprise administration an effective amount of a pharmaceutical composition comprising therapeutic protocells and optionally at least one additional bioactive (e.g., anti-viral) agent.

Diagnostic methods may comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Vaccine Embodiments

Historically, vaccines have worked by eliciting long lived soluble antibody production. These B cell vaccines are capable of neutralizing or blocking the spread of pathogens in the body. This long-lived antibody response primarily targets and neutralizes pathogens as they are spreading from cell to cell, however, they are less effective at eliminating the pathogen once it has entered the host cell. On the other hand, T cell vaccines generate a population of immune cells capable of identifying infected cells and, through affinity dependent mechanisms, kill the cell; thereby eliminating pathogen production at its source. The CD4+ T cells activate innate immune cells, promote B cell antibody production, and provide growth factors and signals for CD8+ T cell maintenance and proliferation. The CD8+ T cells directly recognize and kill virally infected host cells. The ultimate goal of a T cell vaccine is to develop long lived CD8+ memory T cells capable of rapid expansion to combat microbial, e.g., viral, infection.

In some embodiments of a vaccine, a protocell includes a porous nanoparticle core which is made of a material comprising silica, polystyrene, alumina, titania, zirconia, or generally metal oxides, organometallates, organosilicates or mixtures thereof. A porous spherical silica nanoparticle core is used for the exemplary protocells and is surrounded by a supported lipid or polymer bi-layer or multi-layer (multilamellar). Various embodiments provide nanostructures and methods for constructing and using the nanostructures and providing protocells. Porous silica particles are often used and are of varying sizes ranging in size (diameter) from less than 5 nm to 200 nm or 500 nm or more are readily available in the art or can be readily prepared using methods known in the art or alternatively, can be purchased from Melorium Technologies, Rochester, N.Y. SkySpring Nanomaterials, Inc., Houston, Tex., USA or from Discovery Scientific, Inc., Vancouver, British Columbia. Multimodal silica nanoparticles may be readily prepared using the procedure of Carroll et al., 2009. Protocells can be readily obtained using methodologies known in the art. Protocells may be readily prepared, including protocells comprising lipids which are fused to the surface of the silica nanoparticle. See, for example, Liu et al. (2009), Liu et al. (2009), Liu et al. (2009), Lu et al. (1999). Other protocells for use are prepared according to the procedures which are presented in Ashley et al. (2010), Lu et al., (1999), Caroll et al., (2009), and as otherwise presented in the experimental section which follows. Multilamellar protocells may be prepared according to the procedures which are set forth in Moon et al., (2011), among others well known in the art. Another approach would be to hydrate lipid films and bath sonicate (without extrusion) and use polydisperse liposome fusion onto monodisperse cores loaded with cargo.

In some embodiments of the vaccine, the protocells include a core-shell structure which comprises a porous particle core surrounded by a shell of lipid which is often a multi-layer (multilamellar), but may include a single bi-layer (unilamellar), (see Liu et al., 2009). The porous particle core can include, for example, a porous nanoparticle made of an inorganic and/or organic material as set forth above surrounded by a lipid bi-layer. In some embodiments of the vaccine, the porous particle core of the protocells can be loaded with various desired species (“cargo”), especially including plasmid DNA which encodes for a microbial protein such as a bacterial protein, e.g., for a vaccine for tetanus, anthrax, haemophilus, pertussis, diphtheria, cholera, lyme disease, bacterial meningitis, Streptococcus pneumoniae, and typhoid, fungal protein, protist protein, archaea protein or a viral protein (fused to ubiquitin or not) or other microbial antigen (each of which may be ubiquitinylated) and additionally, depending upon the ultimate therapeutic goal, small molecules bioactive agents (e.g., antibiotics and/or anti-cancer agents as otherwise such as adjuvants as described herein), large molecules (e.g., especially including plasmid DNA, other macromolecules such as RNA, including small interfering RNA or siRNA or small hairpin RNA or shRNA or a polypeptide. In certain aspects, the protocells are loaded with super-coiled plasmid DNA, which can be used to deliver the microbial protein or optionally, other macromolecules such as a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anti-microbial agents and/or anti-cancer agents, nucleic acids (DNA and RNA, including siRNA and shRNA and plasmids which, after delivery to a cell, express one or more polypeptides, especially a full length microbial protein, e.g., fused to ubiquitin as a fusion protein or RNA molecules), such as for a particular purpose, as an immunogenic material which may optionally include a further therapeutic application or a diagnostic application.

In some embodiments, the lipid bi-layer of the protocells can provide biocompatibility and can be modified to possess targeting species including, for example, targeting peptides including oligopeptides, antibodies, aptamers, and PEG (polyethylene glycol) (including PEG covalently linked to specific targeting species), among others, to allow, for example, further stability of the protocells and/or a targeted delivery into an antigen presenting cell (APC).

The protocell particle size distribution, according to the vaccine embodiment, depending on the application and biological effect, may be monodisperse or polydisperse. The silica cores can be rather monodisperse (e.g., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol. Polydisperse populations can be sized into monodisperse populations. All of these are suitable for protocell formation. Protocells may be no more than about 500 nm in diameter, e.g., no more than about 200 nm in diameter in order to afford delivery to a patient or subject and produce an intended therapeutic effect. The pores of the protocells may vary in order to load plasmid DNA and/or other macromolecules into the core of the protocell. These may be varied pursuant to methods which are well known in the art.

Protocells according to the vaccine embodiment generally range in size from greater than about 8-10 nm to about 5 μm in diameter, about 20-nm-3 μm in diameter, about 10 nm to about 500 nm, or about 20-200-nm (including about 150 nm, which may be a mean or median diameter) or larger. As discussed above, the protocell population may be considered monodisperse or polydisperse based upon the mean or median diameter of the population of protocells. Size is very important to immunogenic aspects as particles smaller than about 8-nm diameter are excreted through kidneys, and those particles larger than about 200 nm are often trapped by the liver and spleen. Thus, an embodiment focuses in smaller sized protocells for drug delivery and diagnostics in the patient or subject.

Protocells according the vaccine embodiment are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Pore sizes of mesopores range from about 2 to 30 nm or from 20 to 50 nm; they can be monosized or bimodal or graded—they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) may be ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm, e.g., if a templating moiety in the aerosol process is not used. They could also be enlarged to macropores, e.g., those having pores of about 50-nm in diameter.

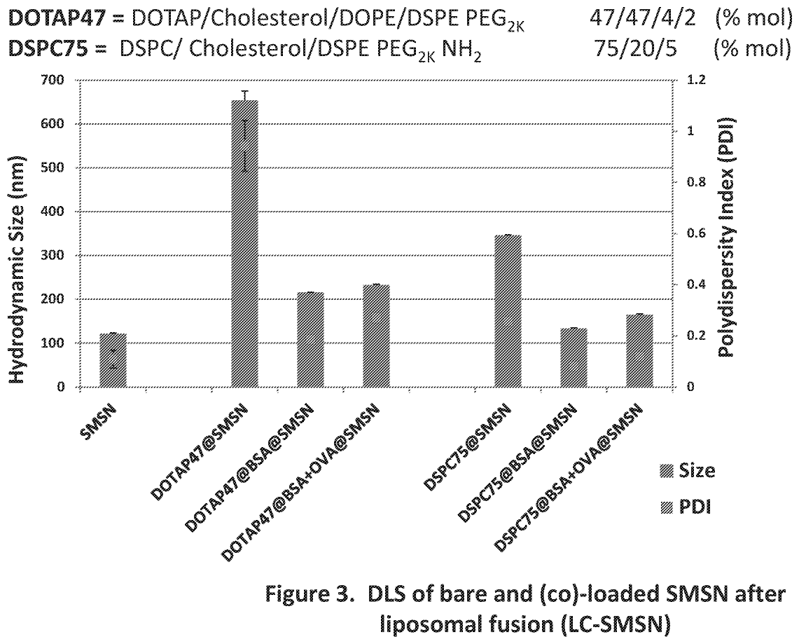

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. See FIG. 3, attached. Attractive electrostatic interactions or hydrophobic interactions control/enhance loading capacity and control release rates. Higher surface areas can lead to higher loadings of drugs/cargos through these attractive interactions, as further explained below.

The surface area of nanoparticles, as measured by the N₂ BET method, ranges from about 100 m²/g to >about 1200 m²/g. In general, the larger the pore size, the smaller the surface area. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to about 50 weight %: defined as (cargo weight/weight of loaded protocell)×100. The optimal loading of cargo is often about 0.01 to 10% but this depends on the drug or drug combination which is incorporated as cargo into the protocell. This is generally expressed in μM of cargo per 10¹⁰ protocell particles with values ranging, for example, from 2000-100 μM per 10¹⁰ particles. Exemplary protocells exhibit release of cargo at pH about 5.5, which is that of the endosome, but are stable at physiological pH of 7 or higher (7.4).

The surface area of the internal space for loading is the pore volume whose value ranges from about 1.1 to 0.5 cubic centimeters per gram (cc/g). Note that in the protocells according to one embodiment, the surface area is mainly internal as opposed to the external geometric surface area of the nanoparticle.

The lipid bi-layer supported on the porous particle according to one embodiment has a lower melting transition temperature, e.g., is more fluid than a lipid bi-layer supported on a non-porous support or the lipid bi-layer in a liposome. This is sometimes important in achieving high affinity binding of targeting ligands at low peptide densities, as it is the bi-layer fluidity that allows lateral diffusion and recruitment of peptides by target cell surface receptors. One embodiment provides for peptides to cluster, which facilitates binding to a complementary target.

In some embodiments, the lipid bi-layer may vary significantly in composition. Ordinarily, any lipid or polymer which is may be used in liposomes may also be used in protocells. Exemplary lipids are as otherwise described herein. Particular lipid bi-layers for use in protocells comprise mixtures of lipids (as otherwise described herein).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bi-layer, the zeta-potential is reduced to between about −10 mV and +5 mV, which is important for maximizing circulation time in the blood and avoiding non-specific interactions.

Depending on how the surfactant template is removed, e.g., calcination at high temperature (500° C.) versus extraction in acidic ethanol, and on the amount of AEPTMS or other silica amine incorporated into the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to the vaccine are that they are stable at pH 7, e.g., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. Quantitative experimental evidence has shown that targeted protocells illicit only a weak immune response in the absence of the components which are incorporated into protocells, because they do not support T-Cell help required for higher affinity IgG, a favorable result.

Various embodiments provide nanostructures which are constructed from nanoparticles which support a lipid bi-layer(s). In embodiments according to the vaccine, the nanostructures may include, for example, a core-shell structure including a porous particle core surrounded by a shell of lipid bi-layer(s). The nanostructure, e.g., a porous silica nanostructure as described above, supports the lipid bi-layer membrane structure.

In some embodiments according to the vaccine, the lipid bi-layer of the protocells can provide biocompatibility and can be modified to possess targeting species including, for example, targeting peptides, antibodies, aptamers, and PEG (polyethylene glycol) linked to targeting species to allow, for example, further stability of the protocells and/or a targeted delivery into a bioactive cell, in particular an APC. PEG, when included in lipid bi-layers, can vary widely in molecular weight (although PEG ranging from about 10 to about 100 units of ethylene glycol, about 15 to about 50 units, about 15 to about 20 units, about 15 to about 25 units, about 16 to about 18 units, etc., may be used and the PEG component which is generally conjugated to phospholipid through an amine group comprises about 1% to about 20%, about 5% to about 15%, or about 10% by weight of the lipids which are included in the lipid bi-layer.

Numerous lipids which are used in liposome delivery systems may be used to form the lipid bi-layer on nanoparticles to provide protocells. Virtually any lipid or polymer which is used to form a liposome or polymersome may be used in the lipid bi-layer which surrounds the nanoparticles to form protocells according to an embodiment. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol is included as a lipid. Often cholesterol is incorporated into lipid bi-layers of protocells in order to enhance structural integrity of the bi-layer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the nanoparticulate cores can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin, a hydrophilic protein, zein, a prolamine, a hydrophobic protein, and copolymers and mixtures thereof.

In still other embodiments, the protocells each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the protocells according to the vaccine can be, for example, mesoporous silica nanoparticles and core-shell nanoparticles. The nanoparticles may incorporate an absorbing molecule, e.g., an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

The cores can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. In some embodiments, the cores have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter. In one embodiment, around 70%, 80%, 90% or 95% of the pores are from around 20 to around 50 nm in diameter. In another embodiment, around 70%, 80%, 90% or 95% of the pores are around 25 to around 40 nm in diameter.

In one embodiment, the cores are synthesized using sol-gel methodology where a silica precursor or silica precursors and a silica precursor or silica precursors conjugated (e.g., covalently bound) to absorber molecules are hydrolyzed in the presence of templates in the form of micelles. The templates are formed using a surfactant such as, for example, hexadecytrimethylammonium bromide (CTAB). It is expected that any surfactant which can form micelles can be used.

In certain embodiments, the core-shell nanoparticles comprise a core and shell. The core comprises silica and an optional absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but are not limited to, isocyanatopropyltriethoxysilane (ICPTS), aminopropyltrimethoxysilane (APTS), mercaptopropyttrimethoxysilane (MPTS), and the like.

In one embodiment, an organosilane (conjugatable silica precursor) used for forming the core has the general formula R₄ₙ SXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

In certain embodiments of the vaccine, the lipid bi-layer is comprised of one or more lipids selected from the group consisting of phosphatidyl-cholines (PCs) and cholesterol.

In certain embodiments, the lipid bi-layer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments: (a) the lipid bi-layer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bi-layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In one embodiment of the vaccine a protocell which is included in compositions may include at least one anti-cancer agent, including an anti-cancer agent which treats a cancer which occurs secondary to a viral infection.

One illustrative embodiment of a protocell of the vaccine comprises a plurality of negatively-charged, nanoporous, nanoparticulate silica cores that: (a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyttriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof; and (b) are encapsulated by and that support a lipid bi-layer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bi-layer comprises a cationic lipid and one or more zwitterionic phospholipids.

Protocells can comprise a wide variety of pharmaceutically-active ingredients.

In certain embodiments, the protocells according to the vaccine may include a reporter for diagnosing a disease state or condition. The term “reporter” is used to describe an imaging agent or moiety which is incorporated into the phospholipid bi-layer or cargo of protocells according to an embodiment and provides a signal which can be measured. The moiety may provide a fluorescent signal or may be a radioisotope which allows radiation detection, among others. Exemplary fluorescent labels for use in protocells (e.g., via conjugation or adsorption to the lipid bi-layer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters are utilized principally in diagnostic applications including diagnosing the existence or progression of a disease state in a patient and or the progress of therapy in a patient or subject.

The term “histone-packaged supercoiled plasmid DNA” is used to describe a component of protocells which utilize an exemplary plasmid DNA which has been “supercoiled” (e.g., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (e.g., complexing DNA with cationic lipid mixtures). In addition, DNA has also been packaged with cationic proteins (including proteins other than histones), as well as gold nanoparticles (e.g., NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins is envisioned. In certain aspects, a combination of human histone proteins H1, H2A, H2B, H3 and H4 in an exemplary ratio of 1:2:2:2:2, although other histone proteins may be used in other, similar ratios, as is known in the art or may be readily practiced. The DNA may also be double stranded linear DNA, instead of plasmid DNA, which also may be optionally supercoiled and/or packaged with histones or other packaging components.

In certain embodiments, protocells comprise a plasmid (which may be a histone-packaged supercoiled plasmid DNA) which encodes a microbial protein, e.g., viral protein, antigen often complexed with ubiquitin protein (e.g., as a fusion protein). The plasmid, including a histone-packaged supercoiled plasmid DNA, may be modified (crosslinked) with a nuclear localization sequence (note that the histone proteins may be crosslinked with the nuclear localization sequence or the plasmid itself can be modified to express a nuclear localization sequence) in order to enhance the ability of the histone-packaged plasmid to penetrate the nucleus of a cell and deposit its contents there (to facilitate expression and ultimately cell death). These peptide sequences assist in carrying the histone-packaged plasmid DNA and the associated histones into the nucleus of a cell to facilitate expression and antigen presentation. Any number of crosslinking agents, well known in the art and as otherwise described herein, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell. In alternative embodiments, the DNA plasmid is included in the absence of histone packaging and/or a nuclear localization sequence and the plasmid expresses a microbial protein (e.g., full length viral protein) in the cytosol of the cell (APC) to which the protocell is delivered.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Exemplary nuclear localization sequences include H₂N-GNQSSNFGPMKGGNFGGRSSGPYGGGGQYFAKPRNQGGYGGC-COOH (SEQ ID NO: 22), RRMKWKK (SEQ ID NO:23), PKKKRKV (SEQ ID NO:24), and KR[PAATKKAGQA]KKKK (SEQ ID NO:25), the NLS of nucleoplasmin, a prototypical bipartite signal comprising two clusters of basic amino acids, separated by a spacer of about 10 amino acids. Numerous other nuclear localization sequences are well known in the art. See, for example, LaCasse et al., 1995; Weis, 1998 and Murat Cokol et al., “Finding nuclear localization signals”, at the website ubic.bioc.columbia.edu/papers/2000 nls/paper.html#tab2.

Viruses that may raise an immunogenic response include any viral bioagent which is an animal virus. Viruses which affect animals, include, for example, Papovaviruses, e.g., polyoma virus and SV40; Poxviruses, e.g., vaccinia virus and variola (smallpox); Adenoviruses, e.g., human adenovirus; Herpesviruses, e.g., Human Herpes Simplex types I and II; Parvoviruses, e.g., adeno associated virus (AAV); Reoviruses, e.g., rotavirus and reovirus of humans; Picomaviruses, e.g., poliovirus; Togaviruses, including the alpha viruses (group A), e.g., Sindbis virus and Semliki forest virus (SFV) and the flaviviruses (group B), e.g., dengue virus, yellow fever virus and the St. Louis encephalitis virus; Retroviruses, e.g., HIV I and II, Rous sarcoma virus (RSV), and mouse leukemia viruses; Rhabdoviruses, e.g., vesicular stomatitis virus (VSV) and rabies virus; Paramyxoviruses, e.g., mumps virus, measles virus and Sendai virus; Arena viruses, e.g., lassa virus; Bunyaviruses, e.g., bunyamwera (encephalitis); Coronaviruses, e.g., common cold, GI distress viruses, Orthomyxovirus, e.g., influenza; Caliciviruses, e.g., Norwalk virus, Hepatitis E virus; Filoviruses, e.g., ebola virus and Marburg virus; and Astroviruses, e.g., astrovirus, among others.

Virus such as Sin Nombre virus, Nipah virus, Influenza (especially H5N1 influenza), Herpes Simplex Virus (HSV1 and HSV-2), Coxsackie virus, Human immunodeficiency virus (I and II), Andes virus, Dengue virus, Papilloma, Epstein-Barr virus (mononucleosis), Variola (smallpox) and other pox viruses and West Nile virus, among numerous others viruses.

A short list of animal viruses which are particularly relevant includes the following viruses: Reovirus, Rotavirus, Enterovirus, Rhinovirus, Hepatovirus, Cardiovirus, Aphthovirus, Parechovirus, Erbovirus, Kobuvirus, Teschovirus, Norwalk virus, Hepatitis E virus, Rubella virus, Lymphocytic choriomeningitis virus, HIV-1, HIV-2, HTLV (especially HTLV-1), Herpes Simplex Virus 1 and 2, Sin Nombre virus, Nipah virus, Coxsackie Virus, Dengue virus, Yellow fever virus, Hepatitis A virus, Hepatitis B virus, Hepatitis C virus, Influenzavirus A, B and C, Isavirus, Thogotovirus, Measles virus, Mumps virus, Respiratory syncytial virus, California encephalitis virus, Hantavirus, Rabies virus, Ebola virus, Marburg virus, Corona virus, Astrovirus, Borna disease virus, and Variola (smallpox virus).

In certain embodiments, compositions may include protocells which contain an anti-cancer agent as a co-therapy, but principally as a separate distinguishable population from immunogenic protocells otherwise described herein. In such an embodiment, protocells which target cancer cells and which contain an anti-cancer agent may be co-administered with immunogenic protocells.

In order to covalently link any of the fusogenic peptides or endosomolytic peptides to components of the lipid bi-layer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, etc.) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition, click chemistry may be used to modify the peptides with azide or alkyne for cu-catalyzed crosslinking, and we could also use a cu-free click chemistry reaction.

The plasmids described herein are used to express a microbial antigen (e.g., a viral protein). Optionally the antigen is in combination with ubiquitin as a fusion protein. In some embodiments, the plasmid vectors are adenoviral, lentiviral and/or retroviral vectors many, of which may readily accommodate the viral protein. Exemplary recombinant adenovirus vectors include those commercialized as the AdEasy™ System by many companies including Stratagene® (stratagene.com), QBiogene® (qbiogene.com), and the ATCC® (atcc. org). AdEasy™ vectors include pShuttle, pShuttle-CMV, and pAdEasy-1. The pAdEasy-1 vector is devoid of E1 and E3 regions so that the recombinant virus will not replicate in cells other than complementing cells, such as human embryonic kidney 293 (HEK293). These methods are described by He et al., Proc. Natl. Acad. Sci., USA, 95, pp. 2509-2514 (1998). An exemplary lentiviral expression system is the The ViraPower™ Lentiviral Expression System (Invitrogen, Carlsbad, California 92008, invitrogen.com) is loosely based on the HIV-1 strain NL4-3. Other commercial adenoviral, lentiviral and retroviral vectors are well known in the art.

The crystal structure of ubiquitin evidences two accessible lysine groups which are used with the crosslinker chemistry described above to anchor the ubiquitin to a component (e.g., viral protein or peptide or a lipid, phospholipid, other) of a lipid bi-layer of the protocell. Ubiquitination does not have to occur in any specific part of the target peptide, it only acts as a marker to signal degradation. This is only intended to speed up the process; the cell would ubiquitinate a foreign peptide naturally delivering ubiquitinated microbial antigens potentially skip this step and speed up the process. Accordingly, ubiquitin is an optional element of the protocells.

As discussed in detail above, the porous nanoparticle core of the vaccine can include porous nanoparticles having at least one dimension, for example, a width or a diameter of about 3000 nm or less, about 1000 nm or less, about 500 nm or less, about 200 nm or less. In one embodiment, the nanoparticle core has an exemplary diameter of about 500 nm or less, e.g., about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, or from 25 nm to 40 nm, although the mean pore size and other properties (e.g., porosity of the porous particle core) are not limited in accordance with various embodiments of the present teachings.

In general, protocells according to the vaccine are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bi-layer(s) as generally described herein.

In the vaccine, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In exemplary aspects, the lipid bi-layer is fused onto the porous particle core to form the protocell. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bi-layer which is used to prepare protocells can be prepared, for example, by extrusion of hydrated lipid films containing other components through a filter with pore size of, for example, about 100 nm, using standard protocols known in the art or as otherwise described herein. The filtered lipid bi-layer films can then be fused with the porous particle cores, for example, by pipette mixing. In certain embodiments, excess amount of lipid bi-layer or lipid bi-layer films can be used to form the protocell in order to improve the protocell colloidal stability.

In various embodiments, the protocell is used in a synergistic system where the lipid bi-layer fusion or liposome fusion (i.e., on the porous particle core) is loaded and sealed with various cargo components with the pores (mesopores) of the particle core, thus creating a loaded protocell useful for cargo delivery across the cell membrane of the lipid bi-layer or through dissolution of the porous nanoparticle, if applicable. In certain embodiments, in addition to fusing a single lipid (e.g., phospholipids) bi-layer, multiple bi-layers with opposite charges can be successively fused onto the porous particle core to further influence cargo loading and/or sealing as well as the release characteristics of the final protocell.

A fusion and synergistic loading mechanism can be included for cargo delivery. For example, cargo can be loaded, encapsulated, or sealed, synergistically through liposome fusion on the porous particles. In addition to microbial proteins, fusion proteins (e.g., viral proteins, including full length viral proteins and fusion proteins based upon viral proteins and ubiquitin) and/or plasmid vectors which can express microbial protein or micrbial protein fused with ubiquitin. The cargo can also include, for example, small molecule drugs (e.g., especially including anti-cancer drugs and/or anti-viral drugs such as anti-HBV or anti-HCV drugs), peptides, proteins, antibodies, DNA (other plasmid DNA, RNAs (including shRNA and siRNA (which may also be expressed by plasmid DNA incorporated as cargo within the protocells), fluorescent dyes, including fluorescent dye peptides which may be expressed by the plasmid DNA incorporated within the protocell as reporters for diagnostic methods associated with establishing the mechanism of immunogenicity of protocells.

Loading of plasmid within the porous core may be difficult to achieve. One approach is to synthesize large pore particles; however, it is somewhat likely that the plasmid will interact with the exterior of the MSNP core regardless of pore size. Therefore, modification of the MSNP framework to incorporate cationic amine groups to form the core as described above will enhance the plasmid/MSNP association due to electrostatic attraction (plasmid carries a net negative charge). Another approach would be to incorporate a small amount of cationic lipids (DOPE, DPPE, DSPE, DOTAP, etc.) into the bi-layer formulation to encourage plasmid/MSNP association.

Protein cargo loading can be electrostatically driven, cationic cores/net negative protein or anionic cores/net positive protein. It is possible to conjugate the protein to the MSNP core using the previously mentioned conjugation strategies by modifying the core with amine, carboxylic acid, thiol, click chemistry, etc. We can also make better use of the pores since protein should be much smaller and more compact than the plasmid constructs. Another approach is to digest the protein into smaller pieces and load the particle with fragments of the protein.

In some embodiments, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively charged species can be loaded as cargo into the pores of a negatively charged silica particle when the lipid bi-layer is fused onto the silica surface showing a fusion and synergistic loading mechanism. In this manner, fusion of a non-negatively charged (i.e., positively charged or neutral) lipid bi-layer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bi-layer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized by the target cell, for example, an APC in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which may be formulated as histone-packaged supercoiled plasmid DNA, e.g., modified with a nuclear localization sequence, can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally modified with a nuclear localization sequence which can localize and concentrate the delivered plasmid DNA into the nucleus of the target cell. As such, loaded protocells can deliver their cargo into targeted cells for therapy or diagnostics.

In various embodiments, the protocells and/or the loaded protocells can provide a targeted delivery methodology for selectively delivering the protocells or the cargo components to targeted cells (e.g., cancer cells). For example, a surface of the lipid bi-layer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In exemplary aspects, the targeting active species is a targeting peptide as otherwise described herein. In certain embodiments, exemplary peptide targeting species include a peptide which targets APC or other cells as otherwise described herein.

For example, by providing a targeting active species (for example, a targeting peptide) on the surface of the loaded protocell, the protocell selectively binds to the targeted cell in accordance with the present teachings. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bi-layer on the porous particle core, the cargo components can be released from the pores of the lipid bi-layer, transported across the protocell membrane of the lipid bi-layer and delivered within the targeted cell. In embodiments, the release profile of cargo components in protocells can be more controllable as compared with when only using liposomes as known in the prior art. The cargo release can be determined by, for example, interactions between the porous core and the lipid bi-layer and/or other parameters such as pH value of the system. For example, the release of cargo can be achieved through the lipid bi-layer, through dissolution of the porous silica; while the release of the cargo from the protocells can be pH-dependent.

In certain embodiments, the pKa for the cargo is often less than 7, or about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the protocell compositions may accommodate immediate release and/or sustained release applications from the protocells themselves.

In certain embodiments, the inclusion of surfactants can be provided to rapidly rupture the lipid bi-layer, transporting the cargo components across the lipid bi-layer of the protocell as well as the targeted cell. In certain embodiments, the phospholipid bi-layer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bi-layer. One example would be gold or magnetic nanoparticles that could use light or heat to generate heat thereby rupturing the bi-layer. Additionally, the bi-layer can be tuned to rupture in the presence of discrete biophysical phenomena, such as during inflammation in response to increased reactive oxygen species production. In certain embodiments, the rupture of the lipid bi-layer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bi-layers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid bi-layer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bi-layer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Exemplary multilamellar liposomes can be produced by the method of Moon, et al., “Interbi-layer-crosslinked multilamellar vesicles as synthetic vaccines for potent humoral and cellular immune responses”, Nature Materials, 2011, 10, pp. 243-251 through crosslinking by divalent cation crosslinking with dithiol chemistry. Another approach would be to hydrate lipid films and bath sonicate (without extrusion) and use polydisperse liposome fusion onto monodisperse cores loaded with cargo.

Pharmaceutical compositions comprise an effective population of protocells as otherwise described herein formulated to effect an intended result (e.g., therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions may also comprise an addition bioactive agent or drug, such as an anti-cancer agent or an anti-microbial agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various mutes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method include humans, companion animals, laboratory animals, and the like. The present disclosure contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, e.g., in unit dosage forms suitable for simple administration of precise dosages.

Pharmaceutical compositions typically include a conventional pharmaceutical carrier or excipient and may additionally include other medicinal agents, carriers, adjuvants, additives and the like. In one embodiment, the composition is about 0.1% to about 85%, about 0.5% to about 75% by weight of a compound or compounds, with the remainder consisting essentially of suitable pharmaceutical excipients.

An injectable composition for parenteral administration (e.g., intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

The composition to be administered will contain a quantity of the selected compound in a pharmaceutically effective amount for therapeutic use in a biological system, including a patient or subject.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g., anti-viral) agent.

Diagnostic methods comprise administering to a patient in need (a patient suspected of having cancer) an effective amount of a population of diagnostic protocells (e.g., protocells which comprise a target species, such as a targeting peptide which binds selectively to APC cells or virus infected cells and a reporter component to indicate the binding of the protocells to APC or virus infected cells if the infection is present) whereupon the binding of protocells to cancer cells as evidenced by the reporter component (moiety) will enable a diagnosis of the existence of cancer in the patient.

An alternative of the diagnostic method can be used to monitor the therapy of cancer or other disease state in a patient, the method comprising administering an effective population of diagnostic protocells (e.g., protocells which comprise a target species, such as a targeting peptide which binds selectively to APC cells or other target cells and a reporter component to indicate the binding of the protocells to the target cells) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### Exemplary Particle Modifications for Hydrophobic Cargo

Porous nanoparticulates used in protocells include mesoporous silica nanoparticles and core-shell nanoparticles. The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

A porous spherical silica nanoparticle may be surrounded by a supported lipid or polymer bilayer or multilayer. Various embodiments provide nanostructures and methods for constructing and using the nanostructures and providing protocells. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll et al., (2009). Protocells can be readily obtained using methodologies known in the art. Protocells may be readily prepared, including protocells comprising lipids which are fused to the surface of the silica nanoparticle. See, for example, Liu et al. (2009), Liu et al. (2009) Lu et al., (1999). In one embodiment, protocells are prepared according to the procedures which are presented in Ashley et al. (2011), Lu et al. (1999), Caroll et al. (2009), and as otherwise presented herein.

One method of making MSNPs is described by Lin et al. (2010) and Lin et al. (2011). In this method, the MSNPs are first produced by standard methods described in the references set forth above by reacting TEOS, TMOS or any other appropriate silane precursor in a surfactant (e.g., CTAB, BDHAC) to produce the MSNPs, which can then be modified with silyhydrocarbon to fully coat the MSNP to form the hydrocarbon coated MSNP. The hydrocarbon coating of the MSNP may be provided prior to a hydrothermal step or after a hydrothermal step by reacting a hydrocarbon silyl chloride (e.g., a mono-, di- or trichloridesilylhydrocarbon) with the MSNP in an appropriate solvent or solvent mixture (e.g., ethanol/chloroform 1:1, cyclohexane, acetonitrile, etc.) at slightly elevated temperature (about 40° C. to about 60° C. until the reaction is complete and the hydrocarbon completely coats the MSMPs (typically about 12 hours or more)). The chlorosilylhydrocarbon is generally used at a molar ratio of at least about 0.5% to about 20%, often about 1% to about 10% (e.g. about 7.5%) to the silica precursor used to form the MSNP in order to ensure that the entire surface of the MSNP is fully coated with the silyl hydrocarbon. Either before or after the coating step, the MSNPs are treated with hydrothermal heating (about 60° C. to about 120° C. in a sealed container for about 12 hours or more). The final MSNPs are fully coated with hydrocarbon by the reaction of SiO groups on the surface of the MSNP with the chlorosilyl groups of the chlorosilyhydrocarbon in order to coat the MSNPs with hydrocarbon through the Si—O—Si bonds which occur at the surface of the MSNP with the silyl groups of the silyl hydrocarbon.

In an alternative embodiment, the MSN after formation (about a 12 hour synthesis using standard methods of preparation, as described above) may be first carboxylated (using a silyl carboxyl agent such as 3-(triethoxysilyl)propylsuccinic anhydride at approximately 0.5% to about 20%, often about 1% to about 15%, often about 1% to about 5%, about 1-1.5% of the TEOS utilized) to form a carboxylic acid group on the surface of the MSN linked to the MSN through Si—O—Si bonds formed when the 3-(triethoxysilyl)propylsuccinic acid and the SiOH groups on the surface of the MSN react. This takes about an hour or so. The carboxylated MSN is then subjected to a hydrothermal step (generally about 12-36 hours, e.g., about 24 hours at an elevated temperature ranging from about 60° C. to about 120° C.) to form a final carboxylated MSN which can be reacted with a crosslinker such as EDC or other crosslinker (the amine portion of the crosslinker forms an amide or other stable bond with the carboxyl group) and the carboxylic/electrophilic end of the linker is reacted with an amine containing phospholipid such as DOPE, DMPE, DPPE or DSPE to form the hydrocarbon coated MSN.

The hydrocarbon coated MSN may then be coated with a phospholipid as described herein to produce hybrid bilayer protocells. In this approach, the hydrocarbon coated MSN is then mixed with a phospholipid which can include a PEGylated phospholipid as otherwise described herein in solvent (chloroform, etc.) and a hydrocarbon/lipophilic cargo and dried together into a film (evaporation, etc.). The film is then hydrated in PBS and washed several times by centrifugation providing hybrid bilayer protocells which have been loaded with a hydrophobic cargo. The hydrocarbon cargo can be a drug, especially an anti-cancer drug, or a hydrophobic reporter for diagnostics.

In some embodiments, the lipid bilayer of the protocells can provide biocompatibility and can be modified to possess targeting species including, for example, targeting peptides including oligopeptides, antibodies, aptamers, and PEG (polyethylene glycol) (including PEG covalently linked to specific targeting species), among others, to allow, for example, further stability of the protocells and/or a targeted delivery into an antigen presenting cell (APC).

The protocell particle size distribution depending on the application and biological effect, may be monodisperse or polydisperse. The silica cores can be rather monodisperse (e.g., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol). Polydisperse populations can be sized into monodisperse populations. All of these are suitable for protocell formation. In some embodiments, protocells are no more than about 500 nm in diameter, or no more than about 200 nm in diameter in order to afford delivery to a patient or subject and produce an intended therapeutic effect. The pores of the protocells may vary in order to load plasmid DNA and/or other macromolecules into the core of the protocell. These may be varied pursuant to methods which are well known in the art.

Hybrid protocells generally range in size from greater than about 8-10 nm to about 5 μm in diameter, about 20-nm-3 μm in diameter, about 10 nm to about 500 nm, or about 20-200-nm (including about 150 nm, which may be a mean or median diameter). In one embodiment, hybrid protocells range in size from about 25 nm up to about 250 nm, e.g., hybrid protocells being less than 200 nm in diameter, less than 150 nm in diameter, or less than about 100 nm in diameter. As discussed above, the protocell population may be considered monodisperse or polydisperse based upon the mean or median diameter of the population of protocells. Size can impact immunogenic aspects as particles smaller than about 8-nm diameter are excreted through kidneys, and those particles larger than about 200 nm are often trapped by the liver and spleen. Thus, an embodiment focuses in smaller sized protocells for drug delivery and diagnostics in the patient or subject.

Protocells are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-nm; they can be monosized or bimodal or graded—they can be ordered or disordered (essentially randomly disposed or worm-like). As noted, larger pores are usually used for loading plasmid DNA and/or full length microbial protein which optionally comprises ubiquitin presented as a fusion protein.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, e.g., 50-nm in diameter.

In an embodiment, the nanostructures include a core-shell structure which comprises a porous particle core surrounded by a shell of lipid such as a bilayer, but possibly a monolayer or multilayer (see Liu et al. (2009)). The porous particle core can include, for example, a porous nanoparticle made of an inorganic and/or organic material as set forth above surrounded by a lipid bilayer. In one embodiment, these lipid bilayer surrounded nanostructures are referred to as “protocells” or “functional protocells,” since they have a supported lipid bilayer membrane structure. In some embodiments, the porous particle core of the protocells can be loaded with various desired species (“cargo”), including small hydrophobic molecules (e.g., anti-cancer agents as otherwise described herein), hydrophobic large molecules, hydrophobic reporters.

In certain embodiments, the cargo components can include, but are not limited to, chemical small molecules for a therapeutic application or a diagnostic application as otherwise disclosed herein.

In some embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

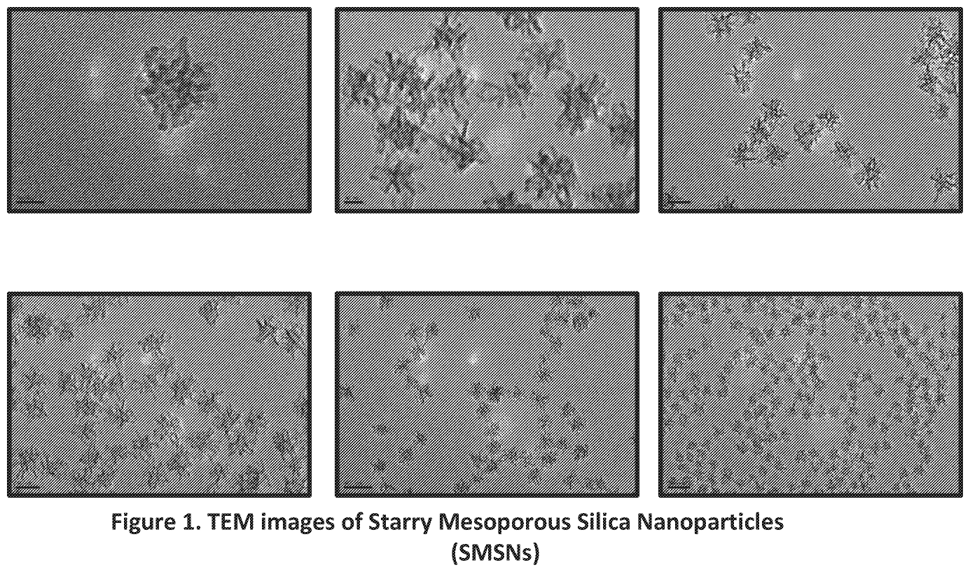

The protocells particle size distribution, depending on the application, may be monodisperse or polydisperse. The silica cores can be rather monodisperse (e.g., a uniform sized population varying no more than about 5% in diameter e.g., ±10-nm for a 200 nm diameter protocell especially if they are prepared using solution techniques) or rather polydisperse (e.g., a polydisperse population can vary widely from a mean or medium diameter, e.g., up to ±200-nm or more if prepared by aerosol. See FIG. 1, attached. Polydisperse populations can be sized into monodisperse populations. All of these are suitable for protocell formation. In one embodiment, protocells may be no more than about 500 nm in diameter, e.g., no more than about 200 nm in diameter, in order to afford delivery to a patient or subject and produce an intended therapeutic effect.

In certain embodiments, protocells generally range in size from greater than about 8-10 nm to about 5 μm in diameter, about 20-nm-3 μm in diameter, about 10 nm to about 500 nm, or about 20-200-nm (including about 150 nm, which may be a mean or median diameter). As discussed above, the protocell population may be considered monodisperse or polydisperse based upon the mean or median diameter of the population of protocells. Size for therapeutic and diagnostic aspects include particles smaller than about 8-nm diameter are excreted through kidneys, and those particles larger than about 200 nm are trapped by the liver and spleen. Thus, an embodiment of focuses in smaller sized protocells for drug delivery and diagnostics in the patient or subject.

In certain embodiments, protocells on are characterized by containing mesopores, e.g., pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. In one embodiment, pore sizes of mesopores range from about 2-30 nm; they can be monosized or bimodal or graded—they can be ordered or disordered (essentially randomly disposed or worm-like).

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2 nm in diameter) all the way down to about 0.03-nm e.g. if a templating moiety in the aerosol process is not used. They could also be enlarged to macropores, e.g., 50 nm in diameter.

Pore surface chemistry of the nanoparticle material can be very diverse—all organosilanes yielding cationic, anionic, hydrophilic, hydrophobic, reactive groups—pore surface chemistry, especially charge and hydrohobicity, affect loading capacity. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100 m2/g to >about 1200 m2/g. In general, the larger the pore size, the smaller the surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO2 or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM per 10¹⁰ particles where we have values ranging from 2000-100 μM per 10¹⁰ particles. In one embodiment, protocells exhibit release of cargo at pH about 5.5, which is that of the endosome, but are stable at physicological pH of 7 or higher (7.4).

The surface area of the internal space for loading is the pore volume whose optimal value ranges from about 1.1 to 0.5 cubic centimeters per gram (cc/g). Note that in certain protocells, the surface area is mainly internal as opposed to the external geometric surface area of the nanoparticle.

The lipid bilayer supported on the porous particle according to one embodiment has a lower melting transition temperature, e.g., is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In one embodiment, the lipid bilayer may vary significantly in composition. Ordinarily, any lipid or polymer which is may be used in liposomes may also be used in protocells. In one embodiment, lipid bilayers for use in protocells comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells are that they are stable at pH 7, e.g., they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Various embodiments provide nanostructures which are constructed from nanoparticles which support a lipid bilayer(s). In some embodiments, the nanostructures include, for example, a core-shell structure including a porous particle core surrounded by a shell of lipid bilayer(s). The nanostructure, e.g., a porous silica nanostructure as described above, supports the lipid bilayer membrane structure.

In some embodiments, the lipid bilayer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20 about 5% to about 15%, or about 10% by weight of the lipids which are included in the lipid bilayer.

Numerous lipids which are used in liposome delivery systems may be used to form the lipid bilayer on nanoparticles to provide protocells. Virtually any lipid or polymer which is used to form a liposome or polymersome may be used in the lipid bilayer which surrounds the nanoparticles to form protocells according to an embodiment. In one embodiment, lipids include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine](DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment given the fact that cholesterol may be an important component of the lipid bilayer of protocells according to an embodiment. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

Pegylated phospholipids include for example, pegylated 1,2-distearoyl-sn-glycero-3-phosphoethanolamine (PEG-DSPE), pegylated 1,2-dioleoyl-sn-glycero-3-phosphoethanolamine (PEG-DOPE), pegylated 1,2-dipalmitoyl-sn-glycero-3-phosphoethanolamine (PEG-DPPE), and pegylated 1,2-dimyristoyl-sn-glycero-3-phosphoethanolamine (PEG-DMPE), among others, including a pegylated ceramide (e.g. N-octanoyl-sphingosine-1-succinylmethoxy-PEG or N-palmitoyl-sphingosine-1-succinylmethoxy-PEG, among others). The PEG generally ranges in size (average molecular weight for the PEG group) from about 350-7500, about 350-5000, about 500-2500, about 1000-2000. Pegylated phospholipids may comprise the entire phospholipid monolayer of hybrid phospholipid protocells, or alternatively they may comprise a minor component of the lipid monolayer or be absent. Accordingly, the percent by weight of a pegylated phospholipid in phospholipid monolayers ranges from 0% to 100% or 0.01% to 99%, e.g., about 5%, 10%, 15%, 20%, 25%, 30%, 35%, 40%, 50%, 55%, 60% and the remaining portion of the phospholipid monolayer comprising at least one additional lipid (such as cholesterol, usually in amounts less than about 50% by weight), including a phospholipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles can be, for example, mesoporous silica nanoparticles and core-shell nanoparticles. The nanoparticles may incorporate an absorbing molecule, e.g. an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be, e.g., from around 5 nm to around 500 nm in size, including all integers and ranges there between. The size is measured as the longest axis of the particle. In various embodiments, the particles are from around 10 nm to around 500 nm and from around 10 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (e.g., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙ SiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known (see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982). The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/538,569, the disclosure of such processes therein are incorporated herein by reference.

In certain embodiments of a protocell, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments: (a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein (b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG).

Protocells can comprise a wide variety of pharmaceutically-active ingredients. The term “hydrophobic drug” or “hydrophobic active agent” is used to describe an active agent which is lipophilic/hydrophobic in nature. Exemplary lipophilic/hydrophobic drugs which are useful include, for example, analgesics and anti-inflammatory agents, such as aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac; Anthelmintics, such as albendazole, bephenium hydroxynaphthoate, cambendazole, dichlorophen, ivermectin, mebendazole, oxamniquine, oxfendazole, oxantel embonate, praziquantel, pyrantel embonate, thiabendazole; Anti-arrhythmic agents such as amiodarone HCl, disopyramide, flecainide acetate, quinidine sulphate; Anti-bacterial agents such as benethamine penicillin, cinoxacin, ciprofloxacin HCl, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim; Anti-coagulants such as dicoumarol, dipyridamole, nicoumalone, phenindione; Anti-depressants such as amoxapine, maprotiline HCl, mianserin HCL, nortriptyline HCl, trazodone HCL, trimipramine maleate; Anti-diabetics such as acetohexamide, chlorpropamide, glibenclamide, gliclazide, glipizide, tolazamide, tolbutamide; Anti-epileptics such as beclamide, carbamazepine, clonazepam, ethotoin, methoin, methsuximide, methylphenobarbitone, oxcarbazepine, paramethadione, phenacemide, phenobarbitone, phenytoin, phensuximide, primidone, sulthiame, valproic acid; Anti-fungal agents such as amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine HCl, terconazole, tioconazole, undecenoic acid; Anti-gout agents such as allopurinol, probenecid, sulphin-pyrazone; Anti-hypertensive agents such as amlodipine, benidipine, darodipine, diitazem HCl, diazoxide, felodipine, guanabenz acetate, isradipine, minoxidil, nicardipine HCl, nifedipine, nimodipine, phenoxybenzamine HCl, prazosin HCL, reserpine, terazosin HCL; Anti-malarials such as amodiaquine, chloroquine, chlorproguanil HCl, halofantrine HCl, mefloquine HCl, proguanil HCl, pyrimethamine, quinine sulphate; Anti-migraine agents such as dihydroergotamine mesylate, ergotamine tartrate, methysergide maleate, pizotifen maleate, sumatriptan succinate; Anti-muscarinic agents such as atropine, benzhexol HCl, biperiden, ethopropazine HCl, hyoscyamine, mepenzolate bromide, oxyphencylcimine HCl, tropicamide; Anti-neoplastic agents and Immunosuppressants such as aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine HCl, tamoxifen citrate, testolactone; Anti-protozoal agents such as benznidazole, clioquinol, decoquinate, diiodohydroxyquinoline, diloxanide furoate, dinitolmide, furzolidone, metronidazole, nimorazole, nitrofurazone, omidazole, tinidazole; Anti-thyroid agents such as carbimazole, propylthiouracil; Anxiolytic, sedatives, hypnotics and neuroleptics such as alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone; n-Blockers such as acebutolol, alprenolol, atenolol, labetalol, metoprolol, nadolol, oxprenolol, pindolol, propranolol; Cardiac Inotropic agents such as amrinone, digitoxin, digoxin, enoximone, lanatoside C, medigoxin; Corticosteroids such as beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone; Diuretics such as acetazolamide, amiloride, bendrofluazide, bumetanide, chlorothiazide, chlorthalidone, ethacrynic acid, frusemide, metolazone, spironolactone, triamterene; Anti-parkinsonian agents such as bromocriptine mesylate, lysuride maleate; Gastro-intestinal agents such as bisacodyl, cimetidine, cisapride, diphenoxylate HCl, domperidone, famotidine, loperamide, mesalazine, nizatidine, omeprazole, ondansetron HCL, ranitidine HCl, sulphasalazine; Histamine H,-Receptor Antagonists such as acrivastine, astemizole, cinnarizine, cyclizine, cyproheptadine HCl, dimenhydrinate, flunarizine HCl, loratadine, meclozine HCl, oxatomide, terfenadine; Lipid regulating agents such as bezafibrate, clofibrate, fenofibrate, gemfibrozil, probucol; Nitrates and other anti-anginal agents such as amyl nitrate, glyceryl trinitrate, isosorbide dinitrate, isosorbide mononitrate, pentaerythritol tetranitrate; Nutritional agents such as betacarotene, vitamin A, vitamin B2, vitamin D, vitamin E, vitamin K; Opioid analgesics such as codeine, dextropropyoxyphene, diamorphine, dihydrocodeine, meptazinol, methadone, morphine, nalbuphine, pentazocine; Sex hormones such as clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone; and Stimulants such as amphetamine, dexamphetamine, dexfenfluramine, fenfluramine, mazindol, among others. Other hydrophobic drugs include rapamycin, docetaxel, paclitaxel, carbazitaxel, thiazolidinediones (e.g. rosiglitazone, pioglitazone, lobeglitazone, troglitazone, netoglitazone, riboglitazone and ciglitazone) and curcumin, among others.

Other targeting peptides are known in the art. Targeting peptides may be complexed or covalently linked to the lipid bilayer through use of a crosslinking agent as otherwise described herein.

In order to covalently link any of the fusogenic peptides or endosomolytic peptides to components of the lipid bilayer, various approaches, well known in the art may be used. For example, the peptides listed above could have a C-terminal poly-His tag, which would be amenable to Ni-NTA conjugation (lipids commercially available from Avanti). In addition, these peptides could be terminated with a C-terminal cysteine for which heterobifunctional crosslinker chemistry (EDC, SMPH, and the like) to link to aminated lipids would be useful. Another approach is to modify lipid constituents with thiol or carboxylic acid to use the same crosslinking strategy. All known crosslinking approaches to crosslinking peptides to lipids or other components of a lipid layer could be used. In addition click chemistry may be used to modify the peptides with azide or alkyne for cu-catalyzed crosslinking, and we could also use a cu-free click chemistry reaction.

Exemplary crosslinking agents include, for example, 1-Ethyl-3-[3-dimethylaminopropyl]carbodiimide hydrochloride (EDC), succinimidyl 4-[N-maleimidomethyl]cyclohexane-1-carboxylate (SMCC), Succinimidyl 6-[β-Maleimidopropionamido]hexanoate (SMPH), N-[β-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

As discussed in detail above, the porous nanoparticle core can include porous nanoparticles having at least one dimension, for example, a width or a diameter of about 3000 nm or less, about 1000 nm or less, about 500 nm or less, about 200 nm or less. In one embodiment, the nanoparticle core is spherical with a diameter of about 500 nm or less, or about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells are biocompatible. Drugs and other cargo components are often loaded by adsorption and/or capillary filling of the pores of the particle core up to approximately 50% by weight of the final protocell (containing all components). In certain embodiments, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In one embodiment, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In certain aspects, the lipid bilayer is fused onto the porous particle core to form the protocell. Protocells can include various lipids in various weight ratios, including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. In one embodiment, the lipid monolayer includes a PEGylated lipid.

The lipid bilayer which is used to prepare protocells can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells such that the plasmid may express one or more fluorescent proteins such as fluorescent green protein or fluorescent red protein which may be used in diagnostic applications.

In various embodiments, the protocell may be used in a synergistic system where the lipid bilayer fusion or liposome fusion (i.e., on the porous particle core) is loaded and sealed with various cargo components with the pores (mesopores) of the particle core, thus lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g., especially including anti-cancer drugs and/or antiviral drugs such as anti-HBV or anti-HCV drugs) and other hydrophobic cargo such as fluorescent dyes.

In other embodiments, the cargo can be loaded into the pores (mesopores) of the porous particle cores to form the loaded protocell. In various embodiments, any conventional technology that js developed for liposome-based drug delivery, for example, targeted delivery using PEGylation, can be transferred and applied to the protocells.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy.

### EXEMPLARY EMBODIMENTS

In one embodiment, a population of starry-like mesoporous silica nanoparticles (SMSNs) is provided. In one embodiment, the SMNS comprises a noble metal. In one embodiment, the SMNSs are surrounded by a lipid layer. In one embodiment, the SMNSs comprise a lipid bi-layer. In one embodiment, the lipid layer is multilamellar. In one embodiment, the SMNSs further comprise one or more cargo molecules. In one embodiment, the lipid layer comprises DOTAP, cholesterol, DSPE, DSPC, DPPC, or any combination hereof. In one embodiment, the lipid layer has about 55% to 65%, 65% to 75%, or 75% to 82% DMPC, about 12% to about 16% 16% to about 21% or about 22% to about 26% mole percent DOTAP, about 13% to about 18%, about 18% to about 23% or 23% to about 28% mole percent cholesterol, about 38% to about 40% or about 40% to about 45% mole percent DPPC, about 1% to about 5%, about 5% to about 8% mole percent DSPE PEG, or any combination thereof. In one embodiment, the pores are at least 25 nm in diameter. In one embodiment, the pores are less than about 40 nm in diameter, or about 20 to about 30 nm in diameter or about 30 to about 40 nm in diameter or about 25 to about 40 nm in diameter. In one embodiment, the pores are about 20 nm to about 50 nm in diameter, or about 30 to about 50 nm in diameter. In one embodiment, the lipid containing nanoparticles are about 125 nm to about 350 nm in diameter. In one embodiment, the lipid containing nanoparticles are about 125 nm to about 250 nm in diameter. In one embodiment, the population exhibits a polydispersity index of less than about 0.6. In one embodiment, the population exhibits a polydispersity index of less than about 0.4. In one embodiment, the nanoparticles are monosized. In one embodiment, the lipid layer comprises more than about 50 mole percent of an anionic, cationic or zwitterionic phospholipid or said lipid bi-layer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid bi-layer comprises cholesterol. In one embodiment, the lipid layer comprises about 0.1 mole percent to about 25 mole percent of at least one lipid comprising a functional group to which a functional moiety may be complexed via coordinated chemistry or covalently attached. In one embodiment, the lipid comprises a functional group such as PEG-containing lipid, optionally wherein said PEG-containing lipid is selected from the group consisting of 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3-phosphoethanolamine-N-[amino(polyethylene glycol)] (DSPE-PEG-NH₂), or a mixture thereof. In one embodiment, the SMSNs comprise at least one component selected from the group consisting of: a cell targeting species; a fusogenic peptide; and a cargo. In one embodiment, the cell targeting species is a peptide, an antibody, an affibody or a small molecule moiety which binds to a cell. In one embodiment,

the cargo is an anti-cancer agent, anti-viral agent, an antibiotic, an antifungal agent, a polynucleotide, a peptide, a protein, an imaging agent, or a mixture thereof. In one embodiment, the polynucleotide comprises encapsulated DNA, double stranded linear DNA, a plasmid DNA, small interfering RNA, small hairpin RNA, microRNA, or mixtures thereof. In one embodiment, the cargo comprises two or more distinct molecules. In one embodiment, the two or more distinct molecules are protein, nucleic acid or protein and nucleic acid. In one embodiment, the cargo comprises CRISPR protein, a plasmid, or siRNA.

The invention will be described by the following non-limiting example.

### Example 1

Synthesis of Monosized Starry Silica Nanoparticles with Utlra Large Mesopores (SMSN).

In a 100 mL glass flask, a mixture of triethanolamine (0.18 g), CTAC (25% in H₂O, 24 mL, pH 6.5) and distilled water (36 mL) was heated to 50° C. and stirred (600 rpm) for 30 min. Directly after, a 20 mL solution of TEOS in cyclohexane (10% v/v) was slowly added to form a biphase system and kept for 16 hours under slow stirring speed (˜250 rpm). The upper organic phase was then removed the bottom aqueous phase (containing the base) was placed in an autoclave at 120° C. for 18-24 h for thermal/basic cooperative etching. Next, the mixture was allowed to cool down, and the nanoparticle suspension was centrifuged. The isolated pellet was resuspended in 15 mL ethanol, centrifuged, and the procedure repeated twice with fresh ethanol. The surfactant removal was achieved by successive washing steps by NH₄NO₃ (6 g/L ethanol, 15 mL) and HCl (1% ethanol, 15 mL, twice); each step included 15 min of sonication followed by centrifugation. All centrifugation cycles were performed at spin rate=50 k rcf for 20 min at room temperature. Store in pure ethanol and quantify.

Control experiments (FIG. 6) were carried out through the following conditions: 1) An ethanolic suspension of template-free nanoparticles was thermally treated at 120° C. for 24 h. The resulting material look exactly like the mother material. 2) An ethanolic suspension of template-free nanoparticles was kept under vacuum for 2 hours to yield nanoparticles powder. The powder was thermally treated at 120° C. for 24 h. The resulting nanoparticles were also similar to the mother particles. 3) An ethanolic suspension of template-free nanoparticles was centrifuged and washed by water. Then the particles were suspended in water (50 mL)-TEA (0.18 g) solution and hydrothermally treated at 120° C. for 24 h. The template free nanoparticles seemed to fuse under such conditions. Thus, the template-filled pores are important to maintain the shape of the particles under basic-thermal conditions.

(co)-Loading of Proteins into SMSNs

Prepare fresh 1 mg/mL solutions of ovalbumin (OVA) and bovine serum albumin (BSA). SMSNs (250 μg) were washed twice by distilled water and suspended to 1 mg/mL. Next, add 250 μL of one or two proteins solutions (1 mg/mL) to the SMSN suspension and mix by pipetting for at least 25 times. Gently shake at room temperature (22° C.) for 15 minutes in the dark.

### Assembly of Liposome/Protein/MSN Nanocomposite

Prepare fresh liposomal suspension of DOTAP- or DSPC-based lipids at 5 mg/mL in PBS (160 mM) by choloroform evaporation and rehydration (PBS) under extensive sonication (45 min).

To the protein-loaded SMSN, add 250 μL of liposomal suspension under sonication (30 seconds) and shake gently for additional 10 minutes at room temperature. Next, the mixture is centrifuged, the supernatants were saved to quantify the loaded extents of proteins. The isolated pellet was washed once by PBS by resuspension/centrifugation and the pellet (lipid-coated protein-loaded SMSN) was finally suspended in PBS. In the absence of protein, the liposomal suspension (250 μL, 5 mg/mL) was directly added to 250 μL (1 mg/mL) SMSN. All other steps were the same.

### Results

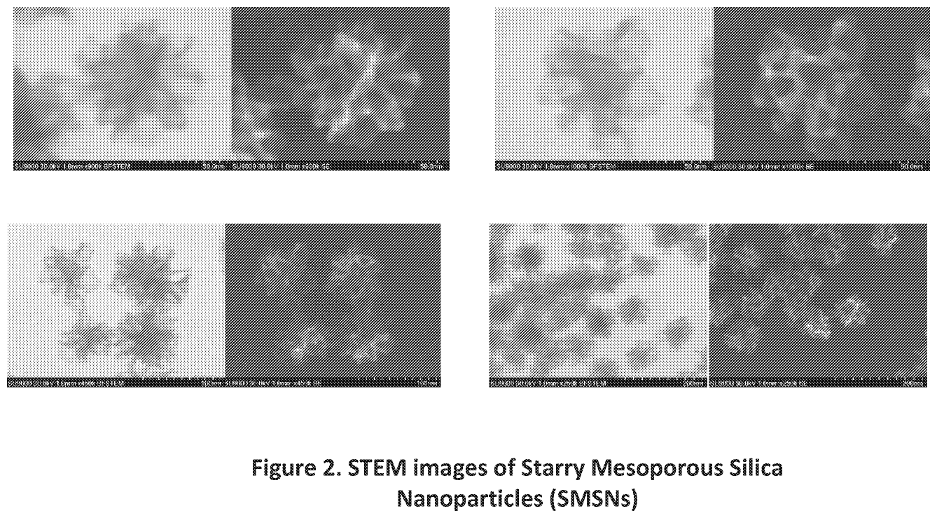

FIG. 1 shows TEM images of starry MSNs (SMSNs) and FIG. 2 shows STEM images of SMSNs.

FIG. 3 illustrates DLS of bare and co-loaded SMSN after liposomal fusion. No fusion on unloaded SMSN indicates that the curvature is not suitable for lipid coating. Fusion on (co)-loaded SMSN indicates that loaded proteins fill the pores thus promoting the fusion. There was an increase of size of LC-SMSN after coloading with 2 proteins.

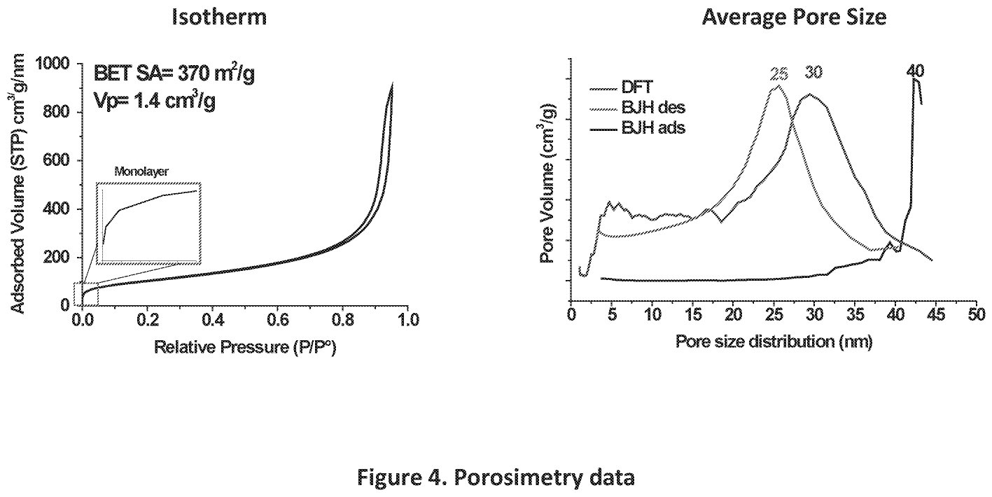

FIG. 4 provides porosimetry data for SMSNs. Isotherm shows monolayer adsorption at very low P/P° (inset). Adsorption occurs on high P/P° (˜0.8). Absence of a saturation plateau plus the hysterisis form indicate large pores. High BET surface area ˜370 m²/g. Pore Size Distribution clearly shows ultra large pores

with average diameter (25-40 nm) depending on the model.

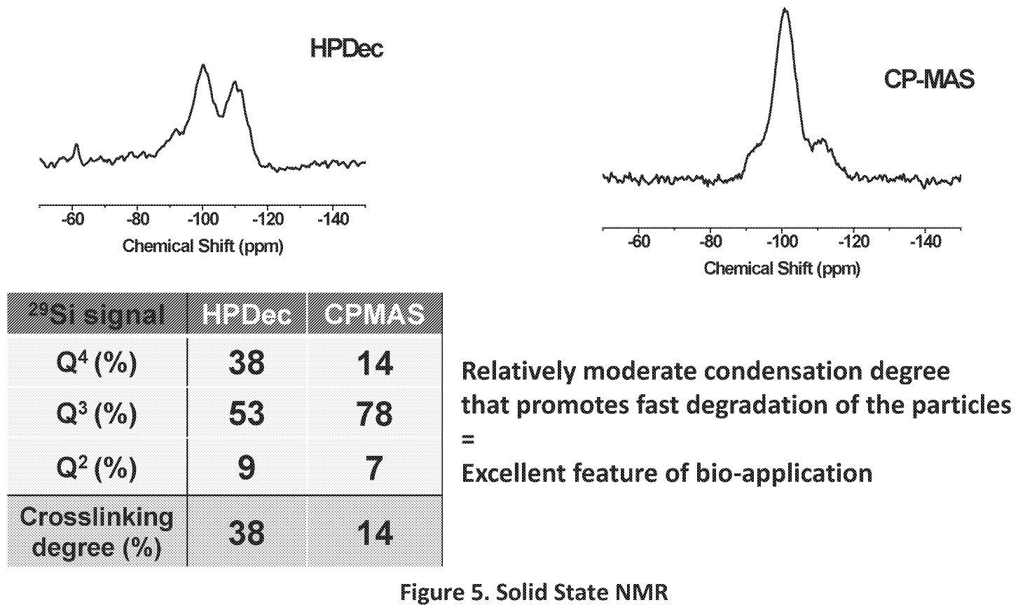

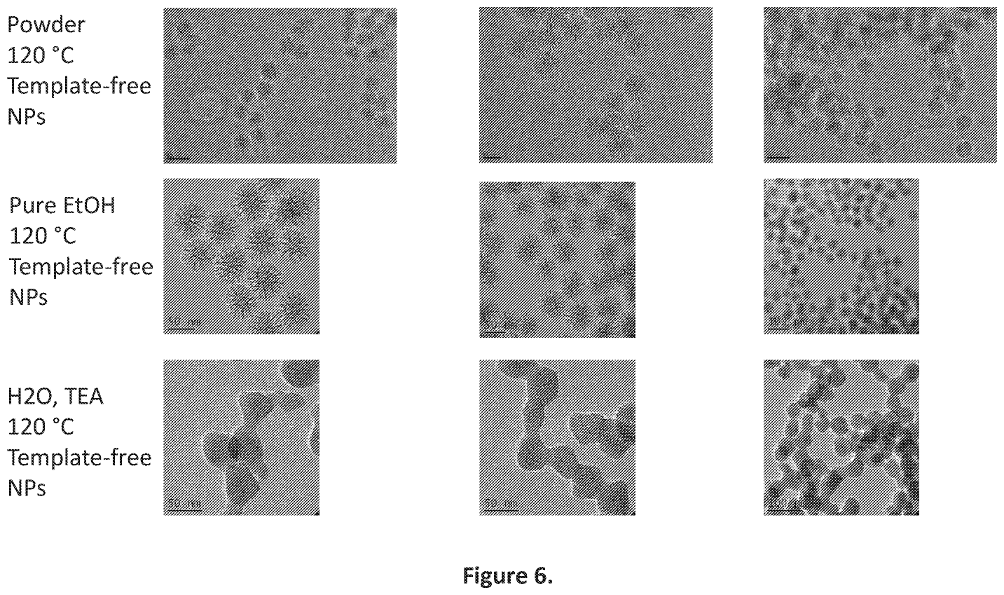

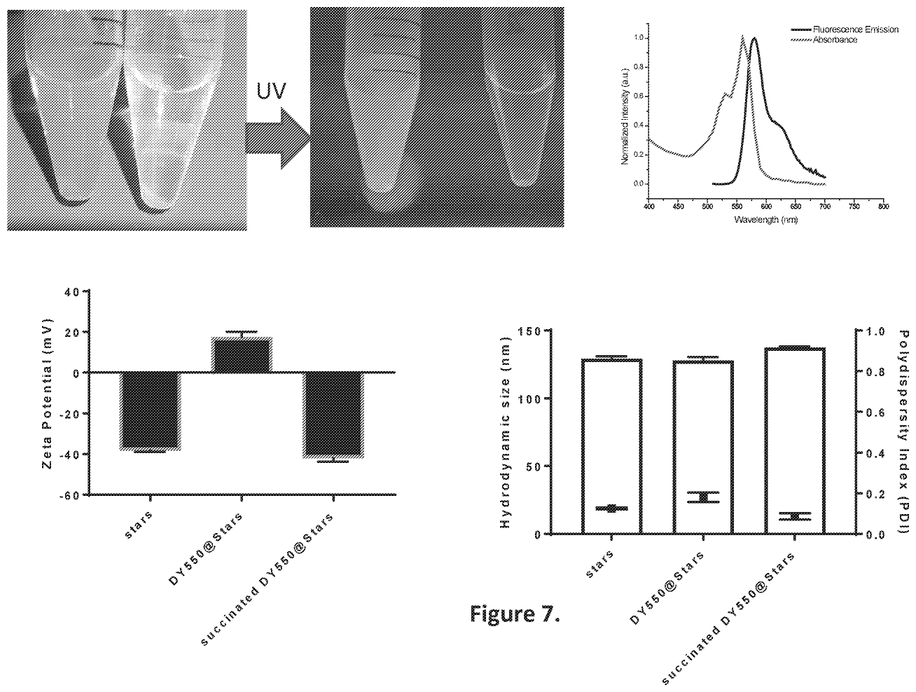

FIG. 5 shows solid state NMR data for SMSNs. There was a relatively moderate condensation degree that promotes fast degradation of the particles.

FIG. 6 shows different conditions tested to determine the parameters for a starry shape.

SMSNs can be derivatized (FIG. 7) and the starry shape is resistant to organic derivatization (FIG. 8).

FIG. 9 provides data showing that succination significantly increases the colloidal stability of SMSNs. Mother nanoparticles dendritic LP8 quickly aggregate in PBS (4 h). Bare SMSNs have low stability in PBS (10 h). Succinated SMSNs extend colloidal stability to more than 3 days. The succinic anhydride reaction with primary amines gives the SMSNs a zwitterionic effect which demonstrates a high stability on bio-relevant media. Other molecules than can give a zwitterionic effect may be anchored on the nanoparticles, molecules such as organosilanes bearing functional groups such as amines and phosphates, amines and phosphonates, or amines and sulfonates. The succination resulted a charge in the particles from highly positive (+20 to +40 mV) to highly negative (−20 to −40 mV) with a straightforward reaction at room temperature. Additionally, by controlling the amount of succinic anhydride/time of reaction the charge can be either controllably reduced or increased without worrying about the grafting efficiency of the aforementioned organosilanes, starting from one parent aminated material.

Lipid coating efficacy of SMSNs depends on cholesterol (FIG. 10). Cholesterol rigidifies the liposomes and makes their fusion on the rough surface of SMSNs more difficult. This is seen by a size and polydispersity increase while increasing the cholesterol extent within the used liposomal formulations.

Control (Stöber particles) shows high hemolysis at 50 μg/mL while SMSNs show no or very low hemolysis at 400-800 μg/mL (FIG. 11).

In the SMSNs, dendritic pores are formed and the surrounding silica matrix is etched in order to increase their size by a cooperative thermal-basic etching. This structure preserves a “nucleus” surrounded by stellate growth of silica matrix and allows for a very homogeneous distribution of cargo molecules within the MSN without any difference between a “hollow core” and a “shell”.

According to the solid state NMR, the process induced a relatively low (40%) condensation degree in comparison to mostly reported systems.

In summary, the silica etching around the pores allows for larger pore sizes. Moreover, homogeneous pores in one SMSN are formed without significant structure differences between the inner and the outer part of a single SMSN. The SMSNs are monodisperse as seen by DLS and TEM, which is usually lost when MSN are exposed to heat, and have a moderate condensation degree responsible for inducing a relatively quick dissolution in bio-relevant media. The SMSNs allow for loading and coloading of different cargo molecules and allow for lipid bilayer/multilayer fusion on loaded SMSNs.

### REFERENCES

Adamson, Cancer J. Clin., 65:212 (2015).
Akbarzadeh et al., Nanoscale Res. Lett., 8:102 (2013).
Allen and Cullis, Science. 303:1818 (2004).
Asefa et al., Chem. Res. Toxicol., 25:2265 (2012).
Ashley et Example al. ACS Nano, 6:2174 (2012).
Ashley et al., Nat. Mater., 10:389 (2011).
Attwood et al., Int. J. Mol. Sci., 14:3514 (2013).
Bae, J. Controlled Release, 133:2 (2009).
Bartlett et al., Proc. Natl. Acad. Sci. U.S.A. 2007, 104, 15549-15554.
Bayer and Bloom, Biophys. J., 58:357 (1990).
Bayu et al., Microporous Mesoporous Mater., 120:447 (2009).
Bertrand et al. Adv. Drug Deliv. Rev., 66:2 (2014).
Blanco et al., Nat. Biotechnol., 33:941 (2015).
Breus et al., ACS Nano, 3:2573 (2009).
Brinker and Scherer, Sol-Gel Science: The Physics and Chemistry of Sol-Gel Processing. Academic press: 2013.
Buranda et al., Lanamuir, 19:1654 (2003).
Butler et al., Protocells: Modular Mesoporous Silica Nanoparticle-Supported Lipid Bi-layers for Drug Delivery. Small 2016.
ça\check{g}das et al., S. Liposomes as Potential Drug Carrier Systems for Drug Delivery. INTECH: 2014.
Carrol et al., Lanamuir, 25:13540 (2009).
Cauda et al., Nano Let., 2484 (2010).
Chauhan et al., Nat. Nanotechnol., 7:383 (2012).
Chen et al., Chem. Mater., 2:4269 (2013).
Choi et al., Nat. Biotechnol., 25:1165 (2007).
Cremer and Boxer, J. Phys. Chem. B, 13:2554 (1999).
Crist et al., Integr. Biol., 5:66 (2013).
Davis et al., Nat Rev Drug Discov., 7:771 (2008).
de Sousa Cavalcante et al., Eur. J. Pharmacol., 741:8 (2014).
Dengler et al., J. Controlled Release, 168:209 (2013).
Deshpande et al., Nanomedicine (London, U. K.), 8:10.2217 (2013).
Dobrovolskaia and McNeil, J. Controlled Release, 172:456 (2013).
Draz et al., Theranostics, 4:872 (2014).
Egusquiaguirre et al., Clin. Transl. Oncol., 14:83 (2012).
Elsabahy and Wooley, Chem. Soc. Rev., 41:2545 (2012).
Epler et al., Adv. Healthcare Mater., 1:348 (2012).
Estephan et al., Lanamuir, 2:18884 (2010).
Farokhzad and Langer, ACS Nano, 2:16. (2009).
Federico et al., Int. J. Nanomed., 7:5423 (2012).
Ferrari, Nat. Nanotechnol., 2:131 (2008).
Garcia-Manyes et al., Biophys. J., 89:1812 (2005).
Han et al., ACS Al. Mater. Interfaces, 1:3342 (2015).
He et al., Small, 7:271 (2011).
Hrkach et al., Sei. Transl. Med., 4:128ra39 (2012).
Huang et al., ACS Nano, 10:648 (2016).
Huang et al., ACS Nano, 5:5390 (2011).
Ii et al., Mol. Cell. Biol., 15:5770 (1995).
Iwamoto, Biol. Pharm. Bull., 36:715 (2013).
Iyer et al., Adv. Drug Delivery Rev., 5:1784 (2013).
Johnson et al., Biophys. J., 59:289 (1991).
Johnson et al., Biophys. J., 83:3371 (2002).
Keller et al., Phys. Rev. Lett., 84:5443 (2000).
Kirk-Othmer, Encyclopedia of Chemical Technology, Vol. 20, 3rd Ed., J. Wiley, N.Y.
Kohli et al., J. Control. Release, 190:274 (2014).
Kresge et al., Nature, 359:710 (1992).
Krishnan and Rajasekaran, Clin. Pharmacol. Ther., 95:168 (2014).
LaCasse et al., Nucl. Acids Res., 2:1647 (1995).
Lammers et al., J. Controlled Release, 161:175 (2012).
Lee et al., Acc. Chem. Res., 44:893 (2011).
Lee et al., Chem. Soc. Rev., 41:2656 (2012).
Leong et al., Nat. Protoc., 5:1406 (2010).
Li et al., Chem. Soc. Rev., 41:2590 (2012).
Liao et al., Interfaces, 2:2607 (2011).
Lin and Haynes, J. Am. Chem. Soc., 132:4834 (2010).
Lin and Haynes, J. Am. Chem. Soc., 4834 (2010)
Lin et al., Chem. Commun., 47:532 (2011).
Lin et al., Chem. Mater., 17:4570 (2005).
Lin et al., J. Am. Chem. Soc., 133:20444 (2011).
Lin et al., J. Phys. Chem. Lett., 2:364 (2012).
Lis et al., Physical Chemistry Chemical Physics, 13:17555 (2011).
Liu et al., ACS Nano, 10:2702 (2016).
Liu et al., Chem. Comm., 5100 (2009).
Liu et al., J. Am. Chem. Soc., 131:1354 (2009a).
Liu et al., J. Am. Chem. Soc., 131:7567 (2009b).
Lu et al., Nature, 398:223 (1999).
Lynch et al. Nano Today. 3:40 (2008).
Mackowiak et al., Nano Lett., 13:2576 (2013).
Markman et al., Adv. Drug Delivery Rev., 65:1866 (2013).
Marsh, CRC Press, (2013).
Meng et al., AC Nano, 5:4434 (2011).
Meng et al., ACS Nano, 9:3540 (2015).
Moon et al., Nat. Mater., 10:243 (2011).
Mornet et al., Nano Lett., 1:281 (2005).
Murat Cokol, Raj Nair & Burkhard Rost, at the website ubic.bioc.columbia.edu/papers/2000 nls/paper.html#tab2.
Nag and Awasthi, Pharmaceutics, 5:542 (2013).
Nandiyanto et al., Microoorous Mesoporous Mater., 120:447 (2009).
Nollert et al., Biophys. J., 69:1447 (1995).
Padera et al., Nature, 427:695 (2004).
Pattni et al., Chem. Rev. Chemical Reviews, 115:10938 (2015).
Peer et al., Nat. Nanotechnol., 2:751 (2007).
Perry et al., Nano Lett., 12:5304 (2012).
Petros and DeSimone, Nat. Rev. Drug Discov., 9:615 (2010).
Pluedemann, Silane Coupling Agents, Plenum Press, N.Y. 1982.
Porotto et al., PloS one, 6:e16874 (2011).
Reviakine and Brisson, Lanamuir, 16:1806 (2000).
Reynolds et al., Toxicol. Appl. Pharmacol., 262:1 (2012).
Richter and Brisson, Biophys. J., 88:3422 (2005).
Riese et al., Mol. Cell. Biol., 15, 5770-5776.
Roggers et al., Mol. Pharm., 5:2770 (2012).
Sackmann, FEBS Lett., 34:3:16 (1994).
Sackmann, Handbook of Biological Physics, 1:213 (1995).
Savarala et al., Lanamuir, 26:12081 (2010).
Schulze et al., Biochimica et Biophysica Acta (BBA)—Molecular Cell Research, 1793:674 (2009).
Seantier and Kasemo, Lanamuir, 25:5767 (2009).
Shen et al., Nano Lett., 14:923 (2014).
Shi et al., Ace. Chem. Res., 4:1123 (2011).
Steichen et al., Eur. J. Pharm. Sci., 48:416 (2013).
Sun et al., Angew. Chem., Int. Ed., 53:12320 (2014).
Tam et al., Acc. Chem. Res., 46:792 (2013).
Torchilin, Nat. Rev. Drug Discovery, 4:145 (2005).
Townson et al., J. Am. Chem. Soc., 135:16030 (2013).
Vargas et al., Adv. Drug Deliv. Rev., 59:1162 (2007).
Vivero-Escoto et al., Small, 6:1952 (2010).
Wang et al., ACS Nano, 4:4371 (2010).
Wang et al., Biomaterials, 34:7662 (2013).
Wang et al., J. Colloid Interface Sci., 385:41 (2012).
Wang et al., RSC Adv., 2:11336 (2012).
Weis, TIBS, 2:185 (1998).
Weis, Trends Biochem. Sci., 2:235 (1998).
Williford et al., Annu. Rev. Biomed. Eng., 16:347 (2014).
Yeagle, CRC press: (2004).
Yu et al., ACS Nano, 5:5717 (2011).
Zarschler et al., Nanoscale, 5:6046 (2014).
Zhang et al., Adv. Funct. Mater., 2:2352 (2014).
Zhang et al., Biomaterials, 35:3650 (2014).
Zhang et al., J. Am. Chem. Soc., 134:15790 (2012).
Zhu et al., Biomacromolecules, 15:1845 (2014).

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

1. A population of starry-like mesoporous silica nanoparticles (SMSNs) optionally surrounded by a lipid layer.

2. The population of claim 1 which comprises a lipid bi-layer.

3. The population of claim 1 which comprises a lipid layer that is multilamellar.

4. The population of claim 1 further comprising one or more cargo molecules.

5. The population of claim 1 wherein the lipid layer comprises DOTAP, cholesterol, DSPE, DSPC, or any combination hereof.

6. The population of claim 1 wherein the pores are at least 25 nm in diameter or wherein the pores are less than about 40 nm in diameter, or about 20 to about 30 nm in diameter or about 30 to about 40 nm in diameter or about 25 to about 40 nm in diameter or wherein the pores are about 20 nm to about 50 nm in diameter, or about 30 to about 50 nm in diameter.

7-8. (canceled)

9. The population of claim 2 wherein the lipid containing nanoparticles are about 125 nm to about 350 nm in diameter.

10. The population of claim 2 wherein the lipid containing nanoparticles are about 125 nm to about 250 nm in diameter.

11. The population of claim 1 which exhibits a polydispersity index of less than about 0.6.

12-13. (canceled)

14. The population of claim 2 wherein the lipid layer comprises more than about 50 mole percent of an anionic, cationic or zwitterionic phospholipid or said lipid bi-layer comprises lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol)(DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and mixtures thereof, or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid bi-layer comprises cholesterol.

15. The population of claim 2, wherein said lipid layer comprises about 0.1 mole percent to about 25 mole percent of at least one lipid comprising a functional group to which a functional moiety may be complexed via coordinated chemistry or covalently attached.

16. (canceled)

17. The population of claim 1, wherein said star-like MSNs comprise at least one component selected from the group consisting of: a cell targeting species; a fusogenic peptide; and a cargo.

18. The population of claim 17, wherein said cell targeting species is a peptide, an antibody, an affibody or a small molecule moiety which binds to a cell.

19. The population of claim 4, wherein said cargo is an anti-cancer agent, anti-viral agent, an antibiotic, an antifungal agent, a polynucleotide, a peptide, a protein, an imaging agent, or a mixture thereof.

20. The population of claim 19, wherein said polynucleotide comprises encapsulated DNA, double stranded linear DNA, a plasmid DNA, small interfering RNA, small hairpin RNA, microRNA, or mixtures thereof.

21. The population of claim 4, wherein said cargo comprises two or more distinct molecules.

22. The population of claim 21 wherein the two or more distinct molecules are protein, nucleic acid or protein and nucleic acid.

23. A pharmaceutical composition comprising a population according to claim 1, and a pharmaceutically acceptable excipient.

24-29. (canceled)

30. A method of inducing immunity to a microbial infection or treating cancer in a mammal, comprising:
administering to the mammal an effective amount of a composition comprising the population of claim 1 which comprises a microbial antigen or an anti-cancer agent.

31. The method of claim 30 further comprising administering an adjuvant.
